SEQUENCE LISTING (14 sequences)

SEQ ID NO: 1 - protein, 265 aa, Homo sapiens
  misc_feature: Incyte ID No 2726173CD1
MTVDSSMSSGYCSLDEELEDCFFTAKTTFFRNAQSKHLSKNVCKPVEETQRPPTLQEIKQKIDSYNTREKNCLGMKLSEDGTYTGFIKVHLKLRRPVTVPAGIRPQSIYDAIKEVNLAATTDKRTSFYLPLDAIKQLHISSTTTVSEVIQGLLKKFMVVDNPQKFALFKRIHKDGQVLFQKLSIADRPLYLRLLAGPDTEVLNFVLKENETGEVEWDAFSIPELQNFLTILEKEEQDKIQQVQKKYDKFRQKLEEALRESQGKPG

SEQ ID NO: 2 - DNA, 3144 bp, Homo sapiens
  misc_feature: Incyte ID No 2726173CB1
ccttgatgcgctggcggcctcggccgggaactccggggtagatgaccgtggacagcagcatgagcagtgggtactgcagcctggacgaggaactggaagactgcttcttcactgctaagactacctttttcagaaatgcgcagagcaaacatctttcaaagaatgtctgtaaacctgtggaggagacacagcgcccgcccacactgcaggagatcaagcagaagatcgacagctacaacacgcgagagaagaactgcctgggcatgaaactgagtgaagacggcacctacacgggtttcatcaaagtgcatctgaaactccggcggcctgtgacggtgcctgctgggatccggccccagtccatctatgatgccatcaaggaggtgaacctggcggctaccacggacaagcggacatccttctacctgcccctagatgccatcaagcagctgcacatcagcagcaccaccaccgtcagtgaggtcatccaggggctgctcaagaagttcatggttgtggacaatccccagaagtttgcactttttaagcggatacacaaggacggacaagtgctcttccagaaactctccattgctgaccgccccctctacctgcgcctgcttgctgggcctgacacggaggtcctcaactttgtgctaaaggagaatgaaactggagaggtagagtgggatgccttctccatccctgaacttcagaacttcctaacaatcctggaaaaagaggagcaggacaaaatccaacaagtgcaaaagaagtatgacaagtttaggcagaaactggaggaggccttaagagaatcccagggcaaacctgggtaaccggtcctgcttcctctcctcctggtgcattcagatttatttgtattattaattattattttgcaacagacactttttctcaggacatctctggcaggtgcatttgtgcctgcccagcagttccagctgtggcaaaagtctcttccatggacaagtgtttgcacgggggttcagctgtgcccgcccccaggctgtgccccaccacagattctgccaaggatcagaactcatgtgaaacaaacagctgacgtcctctctcgatctgcaagcctttcaccaaccaaatagttgcctctctcgtcaccaaactggaacctcacaccagccggcaaaggaaggaagaaaggttttagagctgtgtgttctttctctggctttgattcttctttgagttctcttacttgccacgtacaggaccattatttatgagtgaaaagttgtagcacattccttttgcaggtctgagctaagcccttgaaagcagggtaatgctcataaaaggactgttcccgcggccccaaggtgcctgttgttcacacttaagggaagtttataaagctactggccccagatgctcagggtaaggagcaccaaagctgaggctggctcagagatctccagagaagctgcagcctgccctggccctggctctggccctggcccacattgcacatggaaacccaaaggcatatatctgcgtatgtgtggtacttagtcacatctttgtcaacaaactgttcgtttttaagttacaaatttgaatttaatgttgtcatcatcgtcatgtgtttccccaaagggaagccagtcattgaccatttaaaaagtctcctgctaagtatggaaatcagacagtaagagaaagccaaaaagcaatgcagagaaaggtgtccaagctgtcttcagccttccccagctaaagagcagaggagggcctgggctacttgggttccccatcggcctccagcactgcctccctcctcccactgcgactctgggatctccaggtgctgcccaaggagttgccttgattacagagaggggagcctccaattcggccaacttggagtcctttctgttttgaagcatgggccagacccggcactgcgctcggagagccggtgggcctggcctccccgtcgacctcagtgcctttttgttttcagagagaaataggagtagggcgagtttgcctgaagctctgctgctggcttctcctgccaggaagtgaacaatggcggcggtgtgggagacaaggccaggagagcccgcgttcagtatgggttgagggtcacagacctccctcccatctgggtgcctgagttttgactccaatcagtgataccagaccacattgacagggaggatcaaattcctgacttacatttgcactggcttcttgtttaggctgaatcctaaaataaattagtcaaaaaattccaacaagtagccaggactgcagagacactccagtgcagagggagaaggacttgtaattttcaaagcagggctggttttccaacccagcctctgagaaaccatttctttgctatcctctgccttcccaagtccctcttgggtcggttcaagcccaagcttgttcgtgtagcttcagaagttccctctccgacccaggctgagtccatactgcccctgatcccagaaggaatgctgacccctcgtcgtatgaactgtgcatagtctccagagcttcaaaggcaacacaagctcgcaactctaagatttttttaaaccacaaaaaccctggttagccatctcatgctcagccttatcacttccctccctttagaaactctctccctgctgtatattaaagggagcaggtggagagtcattttccttcgtcctgcatgtctctaacattaatagaaggcatggctcctgctgcaaccgctgtgaatgctgctgagaacctccctctatggggatggctattttatttttgagaaggaaaaaaaaagtcatgtatatatacacataaaggcatatagctatatataaagagataagggtgtttatgaaatgagaaaattattggacaattcagactttactaaagcacagttagacccaaggcctatgctgaggtctaaacctctgaaaaaagtatagtatcgagtacccgttccctcccagaggtgggagtaactgctggtagtgccttctttggttgtgttgctcagtgtgtaagtgtttgtttccaggatattttctttttaaatgtctttcttatatgggttttaaaaaaaagtaataaaagcctgttgcaaaaatgaaaaaaaaa

SEQ ID NO: 3 - DNA, 236 bp, Homo sapiens
  misc_feature: Incyte ID No 2201728H1
ccttgatgngctggcggcctcggccgggaactccggggtagatgaccgtggacagcagcatgancagtgggtactgcagcctggacgaggaactggaagactgcttcttcactgctaagactacctttttcagaaatgcgcagagcaaacatctttcaaagaatgtctgtaaacctgtggaggagacacagcgcccgcccacactgcaggagatcaagcagaagatcgacagctac

SEQ ID NO: 4 - DNA, 510 bp, Homo sapiens
  misc_feature: Incyte ID No 2726173F6
gagaggtagagtgggatgccttctccatccctgaacttcagaacttcctaacaatcctggaaaaagaggagcaggacaaaatccaacaagtgcaaaagaagtatgacaagtttaggcagaaactggaggaggccttaagagaatcccagggcaaacctgggtaaccggtcctgcttcctctcctcctggtgcattcagatttatttgtattattaattattattttgcaacagacactttttctcaggacatctctggcaggtgcatttgtgcctgcccagcagttccagctgtggcaaaagtctcttccatggacaagtgtttgcacgggggttcantgtgcccgcccccaggctgtgccccaccacagatctgccaaggatcagaactcatgtgaacaaacagctgacgtcctctctcgatctgcaagcctttcaaccaaccaaatagttggctctctcgtaaccaaactggaacctcacacccaggccggcaaangaanggangaaa

SEQ ID NO: 5 - DNA, 604 bp, Homo sapiens
  misc_feature: Incyte ID No 1683926F6
tgcctgcccagcagttccagctgtggcaaaagtctcttccatggacaagtgtttgcacgggggttcagctgtgcccgcccccaggctgtgccccaccacagattctgccaaggatcagaactcatgtgaaacaaacagctgacgtcctctctcgatctgcaagcctttcaccaaccaaatagttgcctctctcgtcaccaaactggaacctcacaccagccggcaaaggaaggnagaaaggttttaganctgtgtgttctttctctggcattgattcttctttgagttctcttacttgccacgtanaggacccattatttatgagtgaaaagttgtagcacattccttttgcaggtctgagtaanccctgaaaacagggtaangctcataaaaggatgttcccgcgggcccaaggggcctgttgttnacacttaagggnagnttttaaagctaatggccccaaatgttcaggggaaggngcnccaaagctgaggctgggtccanagatntccaaaaaagttgagcttgccctggcctggntntgggcctggcccanattgccatgggaaccccaagggtanatcngngganntgtggattaanc

SEQ ID NO: 6 - DNA, 568 bp, Homo sapiens
  misc_feature: Incyte ID No 1624086F6
gtggtacttagtcacatctttgtcaacaaactgttcgtttttaagttacaaatttgaatttaatgttgtcatcatcgtcatgtgtttccccaaagggaagccagtcattgaccatttaaaaagtctcctgctaagtatggaaatcagacagtaagagaaagccaaaaagcaatgcagagaaaggtgtccaagctgtcttcagccttccccagctaaagagcagaggagggcctgggctacttgggttccccatcggcctccagcactgcctccctcctcccactgcgactctgggatctccaggtgctgcccaaggagttgccttgattacagagaggggagcctccaattcggccaacttggagtcctttctgttttgaagcatgggccagacccggcatgcgctcggagaccggtgggcctggcctccccgtcgacctcagtgctttttgtttcagagagaaataggagtagggcgagtttgctgaanctctgtgctggcttctcctgccaagaagtgaacaatggcgggcggtgtgggagacaaggccangagagcccgcgttca

SEQ ID NO: 7 - DNA, 757 bp, Homo sapiens
  misc_feature: Incyte ID No 157250R1
accatttaaaagtctcctgctaagtatgggaaatcagacagtaagagaaagccaaaaagcaatgcagaggaaaggtgtccaagctgtcttcagccttccccagctaaagagcagaggagggcctgggctacttgggttccccatcggcctccagcactgcctccctcctcccactgcgactctgggatctccaggtgctgcccaaggagttgccttgattacagagaggggagcctncaattcggccaacttggagtcctttctgttttgaagcatgggccagacccggcactgcgctcggagagccggtgggcctgggcttcccgtcgacctcagtgcctttttgttttcagagagaaataggagtagggcgagtttgcctgaagctctgctgctngcttctcctgccaggaagtgaacaatggcggcggtgttggagacaaggccaggagagcccgngttcaatattggttnagggtcanaagaccttccttccaatttggtgcctnagttttnacttccaattcaagtgaattaccagaccacaatttacaaggagggtcaaaattctgacttanatttnaattgnttnttgtttaaggtnaatccnaaataaantaggnaaaaaattccacaagtagccaggcttnaagggcaattcagtgcaagagggagaaggccttntaatttnaaaggagggntggttttcaacccagccttttngaaccattttttnnnatccnnnccttccaaagcc

SEQ ID NO: 8 - DNA, 787 bp, Homo sapiens
  misc_feature: Incyte ID No 157250F1
cngtttttttttcatttttgcaacaggcttttattacttttttttaaaacccatataagaaagacatttaaaaagaaaatatcctggaaacaaacacttacacactgagcaacacaaccaaagaaggcactaccagcagttactcccacctctgggagggaacgggtactcgatactatacttttttcagaggtttagacctcagcataggccttgggtctaactgtgctttagtaaagtctgaattgtccaataattttctcatttcataaacacccttatctctttatatatagctatatgcctttatgtgtatatatacatgactttttttttccttctcaaaaataaaatagccatccccatagagggaggttctcagcagcattcacagcggttgcagcaggagccatgccttctattaatgttagagacatgcaggacgaaggaaaatgactctccacctgctccctttaatatacagcagggagagagtttctaaagggagggaagtgataaggctgaggcatgaggatggctaaccagggtttttgtggtttaaaaaaatcttagngttgcgagcttgtgttgcctttgaagctctggagactatgcacagttcatacgacgnggggtcagcattncttctgggntcaggggcagttngnctnagctgggtcgggaggggactttgaagcncacggacaagtttggcttgaccgncccagngggcttgggaggcagggntgcaagaatggtntagggctgggttggaaacagcctnttt

SEQ ID NO: 9 - DNA, 489 bp, Homo sapiens
  misc_feature: Incyte ID No 1208878R1
agaaactctctccctgctgtatattaaagggagcaggtggagagtcattttccttcgtcctgcatgtctctaacattaatagaaggcatggctcctgctgcaaccgctgtgaatgctgctgagaacctccctctatggggatggctattttattttngagaaggaaaaaaaaagtcatgtatatatacacataaaggcatatagctatatataaagagataagggtgtttatgaaatgagaaaattattggacaattcagactttactaaagcacagttagacccaaggcctatgctgaggtctaaacctctgaaaaaagtatagtatcgagtacccgttccctcccagaggtgggagtaactgctggtagtgccttctttggttgtgttgctcagtgtgtaagtgtttgtttccaggatattttctttttaaatgtctttcttatatgggttttaaaaaaaagtaataaaagcctgttgcaaaaatga

SEQ ID NO: 10 - DNA, 278 bp, Rattus norvegicus
  misc_feature: Incyte ID No 700541810H1
gcaaaagcaccatgagctagcgactctgaggctgtgcatgccatagcccctggctccctgccgcctcaggctcagccttacactcccctcccttgagaagtcccctttctgctgtgtattacagggacatacggaaactcattcccttcatcctgcatgtgtgtagcattaggagaaggcatggctcctgctgcaaacactgtgaatgctgctcagacctccctccatggatggctattttatttttgacaagaaaaaaaattcatgtatatataaaa

SEQ ID NO: 11 - DNA, 220 bp, Rattus norvegicus
  misc_feature: Incyte ID No 700632515H1
aagctgtgcaggcgcttgaaaccccaatgaaggaagcacggactgcctgcctgagagcagcctggtaacccactagaacccgcacctgagccagaacccgaagccccctcgcgcacccctgccccgcagcagtccgatgagctcagactcgggcggggcccaatgaccgtagacagcagcatgagcagcgggtactgcagcctggatgaggaactggaag

SEQ ID NO: 12 - DNA, 214 bp, Rattus norvegicus
  misc_feature: Incyte ID No 700540004H1
aaaatgtatatacatatatatgtatatacacatagaggcatatagctatatataaagaaaaggtgtttattcagaaagggatgatcaacttcagactttaaagcacagttagacccgggacctctactgaggttgagccgctggaaacgtggagttccttaagtctcagaggggtctaactgctggtagtgccttctatggttgtatggctctg

SEQ ID NO: 13 - protein, 413 aa, Rattus norvegicus
  misc_feature: Incyte ID No g2459833
MASPAIGQRPYPLLLDPEPPRYLQSLGGTEPPPPARPRRCIPTALISASGASEGRGSRRNARGDPEPTPRDCRHARPVRPGLQQRLRRRPGSHRPRDVRSIFEQPQDPRVLAERGEGHRFAELALRGGPGWCDLCGREVLRQALRCANCKFTCHPECRSLIQLDCRQKEGPALDRQSPESTLTPTFNKNVCKAVEETQHPPTIQEIKQKIDSYNSREKHCLGMKLSEDGTYTGFIKVHLKLRRPVTVPAGIRPQSIYDAIKEVNPAATTDKRTSFYLPLDAIKQLHISSSTTVSEVIQGLLKKFMVVDNPQKFALFKRIHKDGQVLFQKLSIADCPLYLRLLAGPDTDVLSFVLKENETGDVEWDAFSIPELQNFLTILEKEEQDKIHQLQKKYNKFRQKLEEALRESQGKPG

SEQ ID NO: 14 - protein, 413 aa, Mus musculus
  misc_feature: Incyte ID No g2997698
MASPAIGQRPYPLLLDPEPPRYLQSLGGTEPPPPARPRRCIPTALIPAAGASEDRGGRRSGRRDPEPTPRDCRHARPVRPGLQPRLRLRPGSHRPRDVRSIFEQPQDPRVLAERGEGHRFVELALRGGPGWCDLCGREVLRQALRCANCKFTCHSECRSLIQLDCRQKGGPALDRRSPGSTLTPTLNQNVCKAVEETQHPPTIQEIKQKIDSYNSREKHCLGMKLSEDGTYTGFIKVHLKLRRPVTVPAGSGPSPSMDAIKEVNPAATTDKRTSFYLPLDAIKQLHISSTTTVSEVIQGLLKKFMVVDNPQKFALFKRIHKDGQVLFQKLSIADYPLYLRLLAGPDTDVLSFVLKENETGEVEWDAFSIPELQNFLTILEKEEQDKIHQLQKKYNKFRQKLEEALRESQGKPG

(19) United States
(12) Patent Application Publication
(10) Pub. No.: US 2003/0099995 A1
(43) Pub. Date: May 29, 2003

(54) Ras association domain containing protein

(75) Inventors: Michael G. Walker, Sunnyvale, CA (US); Tod M. Klinger, San Carlos, CA (US); Randi E. Krasnow, Stanford, CA (US)

Correspondence Address: LEGAL DEPARTMENT, INCYTE GENOMICS, INC., 3160 PORTER DRIVE, PALO ALTO, CA, 94304, US

(73) Assignee: Incyte Genomics, Inc., Palo Alto, CA

(21) Appl. No.: 10/270,845

(22) Filed: Oct. 10, 2002

Related U.S. Application Data
(63) Continuation-in-part of application No. 09/614,069, filed on Jul. 11, 2000, now Pat. No. 6,485,910.
Application No. 09/614,069 is a continuation-in-part of application No. 09/195,292, filed on Nov. 18, 1998, now abandoned.
Application No. 09/195,292 is a continuation-in-part of application No. 09/023,655, filed on Feb. 9, 1998.

(51) Int. Cl. C12Q 1/68; G01N 33/53; G01N 33/567; C07H 21/04; C12N 9/10; C12P 21/02; C12N 5/06; A61K 38/17

(52) U.S. Cl. 435/6; 435/7.2; 435/69.1; 435/193; 435/320.1; 435/325; 514/12; 536/23.2

(57) ABSTRACT

The invention provides a Ras association domain containing protein, its encoding mammalian cDNA, and an antibody that specifically binds the protein. It also provides for the use of the cDNAs, complements, and variants thereof and of the protein, portions thereof and antibodies thereto to diagnose, stage, treat or monitor the progression or treatment of cell proliferative and inflammatory disorders.

This application is a continuation-in-part of copending U.S. Ser. No. 09/614,069 filed Jul. 11, 2000, U.S. Ser. No. 09/195,292 filed Nov. 18, 1998, and U.S. Ser. No. 09/023,655 filed Feb. 9, 1998; all of which are incorporated by reference herein.

### FIELD OF THE INVENTION

This invention relates to a Ras association domain containing protein, its encoding cDNA and an antibody that specifically binds the protein; all of which can be used to diagnosis, stage, treat or monitor the progression or treatment of cell proliferative and inflammatory disorders.

### BACKGROUND OF THE INVENTION

Signal transduction is the general process by which cells respond to extracellular signals. In typical signal transduction pathways, binding of a signaling molecule such as a hormone, neurotransmitter, or growth factor to a cell membrane receptor is coupled to the action of an intracellular second messenger. G protein-coupled receptors (GPCRs) control intracellular processes through the activation of guanine nucleotide-binding proteins (G proteins). G proteins are heterotrimeric and consist of α, β, and γ subunits. The α subunit contains a guanine nucleotide binding domain and has GTPase activity. When GTP binds to the α subunit, it dissociates from the β and γ subunits and interacts with cellular target molecules. Hydrolysis of GTP to GDP serves as a molecular switch controlling the interactions of the α subunit with other proteins. The GDP bound form of the α subunit dissociates from its cellular target and reassociates with the β and γ subunits. A number of accessory proteins modulate G protein function by controlling their nucleotide state or membrane association. These regulatory molecules include exchange factors (GEFs) which stimulate GDP-GTP exchange, GTPase activating proteins (GAPs) which promote GTP hydrolysis, and guanine nucleotide dissociation inhibitors (GDIs) which inhibit guanine nucleotide dissociation and stabilize the GDP-bound form. G proteins can be classified into at least five subfamilies: Ras, Rho, Ran, Rab, and ADP-ribosylation factor, and they regulate various cell functions including cell growth and differentiation, cytoskeletal organization, and intracellular vesicle transport and secretion.

The Ras subfamily transduces signals from tyrosine kinase receptors, non-tyrosine kinase receptors, and heterotrimeric GPCRs (Fantl et al. (1993) Annu Rev Biochem 62:453-481; Woodrow et a. (1993) J Immunol 150:3853-3861; and Van Corven et al. (1993) Proc Natl Acad Sci 90:1257-1261). Stimulation of cell surface receptors activates Ras which, in turn, activates cytoplasmic kinases that control cell growth and differentiation. The first Ras targets identified were the Raf kinases (Avruch et al. (1994) Trends Biochem Sci 19:279-283). Interaction of Ras and Raf leads to activation of the MAP kinase cascade of serine/threonine kinases, which activate key transcription factors that control gene expression and protein synthesis (Barbacid (1987) Ann Rev Biochem 56:779-827; Treisman (1994) Curr Opin Genet Dev 4:96-101). Mutant Ras proteins, which bind but do not hydrolyze GTP, are constitutively activated, and cause continuous cell proliferation and cancer (Bos (1989) Cancer Res 49:4682-4689; Grunicke and Maly (1993) 4:389-402).

Ras regulates other signaling pathways by direct interaction with different cellular targets (Katz and McCormick (1997) Curr Opin Genet Dev 7:75-79). One such target is RalGDS, a guanine nucleotide dissociation stimulator for the Ras-like GTPase, Ral (Albright et al. (1993) EMBO J. 12:339-347). RalGDS couples the Ras and Ral signaling pathways. Epidermal growth factor (EGF) stimulates the association of RalGDS with Ras in mammalian cells, which activates the GEF activity of RalGDS (Kikuchi and Williams (1996) J Biol Chem 271:588-594; Urano et al. (1996) EMBO J. 15:810-816). Ral activation by RalGDS leads to activation of Src, a tyrosine kinase that phosphorylates other molecules including transcription factors and components of the actin cytoskeleton (Goi et al. (2000) EMBO J. 19:623-630). Ral interacts with a number of signaling molecules including Ral-binding protein, a GAP for the Rho-like GTPases; Cdc42 and Rac, which regulate cytoskeletal rearrangement; and phospholipase D1, which is involved in vesicular trafficking (Feig et al. (1996) Trends Biochem Sci 21:438-441; Voss et al. (1999) J Biol Chem 274:34691-34698).

Nore1 was identified from a yeast two-hybrid screen as a protein that interacts with Ras and Ras-related protein, Rap1β (Vavvas et al. (1998) J Biol Chem 273:5439-5442). It is a highly basic protein %(pI=9.4) of 413 amino acids that contains a cysteine-histidine-rich region predicted to be a diacylglycerol/phorbol ester binding site, a proline-rich region at its N-terminus that may be an SH3 binding domain, and a Ras/Rap binding domain located at its C-terminus. Nore1 binds Ras in vitro in a GTP dependent manner. Experiments in vivo show that the association of Nore1 with Ras is dependent on EGF and 12-O-tetradecanoylphorbol-13-acetate activation in COS-7 cells and on EGF in KB cells.

Ras and other G proteins play roles in regulating the immune inflammatory response. Granulocytes, which include basophils, eosinophils, and neutrophils, play critical roles in inflammation. Eosinophils release toxic granule proteins, which kill microorganisms, and secrete prostaglandins, leukotrienes and cytokines, which amplify the inflammatory response. They sustain inflammation in allergic reactions and their malfunction can cause asthma and other allergic diseases. Interleukin-5 is a cytokine that regulates the growth, activation, and survival of eosinophils. The signal transduction mechanism of IL-5 in eosinophils involves the Ras-MAP kinase and Jak-Stat pathways (Pazdrak et al. (1995) J Exp Med 181:1827-1834; Adachi and Alam (1998) Am J Physiol 275:C623-633). Raf-1 kinase activation by Ras is implicated in eosinophil degranulation.

Neutrophils migrate to inflammatory sites where they eliminate pathogens by phagocytosis and release toxic products from their granules that kill microorganisms. G proteins, including Ras, Ral, Rac1 and Rap1 regulate neutrophil function (M'Rabet et al. (1999) J Biol Chem 274:21847-21852). Rac1 may be involved in the respiratory burst of neutrophils. Ras and Rap1 are activated in response to the chemotactic agent, formyl methionine leucine phenylalanine (fMLP); the lipid mediator, platelet activating factor (PAF); and the cytokine, granulocyte-macrophage colony-stimulating factor (GM-CSF). Both Ras and Rap1 appear to play roles in neutrophil activation. Ral is activated by fMLP and PAF, but not by GM-CSF, and may be involved in chemotaxis, phagocytosis or degranulation. Impairment of neutrophil function is associated with various inflammatory and autoimmune disorders.

The discovery of a cDNA encoding a new Ras target or effector and an antibody that specifically binds the protein satisfies a need in the art by providing compositions which can be used to diagnose, stage, treat or monitor the progression or treatment of cell proliferative and inflammatory disorders.

### SUMMARY OF THE INVENTION

The invention is based on the discovery of a Ras association domain containing protein (RADCP), its encoding cDNA, and an antibody that specifically binds the protein which can be used to diagnose, stage, treat or monitor the progression or treatment of cell proliferative and inflammatory disorders, particularly allergies, asthma, hypereosinophilia, cancers such as prostate adenofibromatous hyperplasia, thymus hyperplasia and lung tumors.

The invention provides an isolated cDNA comprising a nucleic acid sequence encoding a protein having the amino acid sequence of SEQ ID NO:1. The invention also provides an isolated cDNA and the complement thereof selected from a nucleic acid sequence of SEQ ID NO:2; a fragment of SEQ ID NO:2 selected from SEQ ID NOs:3-9; an oligonucleotide extending from about nucleotide 1 to about nucleotide 50 of SEQ ID NO:2, and a homolog of SEQ ID NO:2 selected from SEQ ID NOs:10-12. The invention further provides a probe consisting of a polynuclotide the hybridizes to the cDNA encoding RADCP.

The invention provides a cell transformed with the cDNA encoding RADCP, a composition comprising the cDNA encoding RADCP and a labeling moiety; a probe comprising the cDNA encoding RADCP, an array element comprising the cDNA encoding RADCP and a substrate upon which the cDNA encoding RADCP is immobilized. The composition, probe, array element or substrate can be used in methods of detection, screening, and purification. In one aspect, the probe is a single-stranded complementary RNA or DNA molecule.

The invention provides a vector containing the cDNA encoding RADCP, a host cell containing the vector, and a method for using the host cell to make RADCP, the method comprising culturing the host cell under conditions for expression of the protein and recovering the protein so produced from host cell culture. The invention also provides a transgenic cell line or organism comprising the vector containing the cDNA encoding RADCP.

The invention provides a method for using a cDNA encoding RADCP to detect the differential expression of a nucleic acid in a sample comprising hybridizing a probe to the nucleic acids, thereby forming hybridization complexes and comparing hybridization complex formation with a standard, wherein the comparison indicates the differential expression of the cDNA in the sample. In one aspect, the method of detection further comprises amplifying the nucleic acids of the sample prior to hybridization. In a second aspect, the sample is selected from blood, lung, lymph node, prostate, spleen, tonsil, and thymus. In a third aspect, comparison to standards is diagnostic of a cell proliferative or inflammatory disorder.

The invention provides a method for using a cDNA to screen a library or plurality of molecules or compounds to identify at least one ligand which specifically binds the cDNA, the method comprising combining the cDNA with the molecules or compounds under conditions to allow specific binding and detecting specific binding to the cDNA, thereby identifying a ligand which specifically binds the cDNA. In one aspect, the molecules or compounds are selected from antisense molecules, branched nucleic acids, DNA molecules, peptides, proteins, RNA molecules, and transcription factors. The invention also provides a method for using a cDNA to purify a ligand which specifically binds the cDNA, the method comprising attaching the cDNA to a substrate, contacting the cDNA with a sample under conditions to allow specific binding, and dissociating the ligand from the cDNA, thereby obtaining purified ligand. The invention further provides a method for assessing efficacy or toxicity of a molecule or compound comprising treating a sample containing nucleic acids with the molecule or compound; hybridizing the nucleic acids with the cDNA encoding RADCP under conditions for hybridization complex formation; determining the amount of complex formation; and comparing the amount of complex formation in the treated sample with the amount of complex formation in an untreated sample, wherein a difference in complex formation indicates the efficacy or toxicity of the molecule or compound.

The invention provides purified RADCP. The invention also provides antigenic epitopes extending from about residue F30 to about residue M75 of SEQ ID NO:1. The invention additionally provides a biologically active peptides extending from about residue A119 to about residue T211 of SEQ ID NO:1. The invention also provides a composition comprising the purified protein and a pharmaceutical carrier, a composition comprising the protein and a labeling moiety, a substrate upon which the protein is immobilized, and an array element comprising the protein. The invention further provides a method for detecting expression of a protein having the amino acid sequence of SEQ ID NO:1 in a sample, the method comprising performing an assay to determine the amount of the protein in a sample; and comparing the amount of protein to standards, thereby detecting expression of the protein in the sample. The invention still further provides a method for diagnosing cancer comprising performing an assay to quantify the amount of the protein expressed in a sample and comparing the amount of protein expressed to standards, thereby diagnosing a cell proliferative or inflammatory disorder. In a one aspect, the assay is selected from antibody or protein arrays, enzyme-linked immunosorbent assays, fluorescence-activated cell sorting, spatial immobilization such as 2D-PAGE and scintillation counting, high performance liquid chromatography or mass spectrophotometry, radioimmunoassays, and western analysis. In a second aspect, the sample is selected from blood, lung, lymph node, prostate, spleen, tonsil, and thymus.

The invention provides a method for using a protein to screen a library or a plurality of molecules or compounds to identify at least one ligand, the method comprising combining the protein with the molecules or compounds under conditions to allow specific binding and detecting specific binding, thereby identifying a ligand which specifically binds the protein. In one aspect, the molecules or compounds are selected from agonists, antagonists, DNA molecules, small drug molecules, immunoglobulins, inhibitors, mimetics, multispecific molecules, peptides, pharmaceutical agents, proteins, and RNA molecules. In another aspect, the ligand is used to treat a subject with a cell proliferative or inflammatory disorder. The invention also provides an therapeutic antibody that specifically binds the protein having the amino acid sequence of SEQ ID NO:1. The invention further provides an antagonist which specifically binds the protein having the amino acid sequence of SEQ ID NO:1. The invention yet further provides a small drug molecule which specifically binds the protein having the amino acid sequence of SEQ ID NO:1. The invention also provides a method for testing ligand for effectiveness as an agonist or antagonist comprising exposing a sample comprising the protein to the molecule or compound, and detecting agonist or antagonist activity in the sample.

The invention provides a method for using a protein to screen a plurality of antibodies to identify an antibody that specifically binds the protein comprising contacting a plurality of antibodies with the protein under conditions to form an antibody:protein complex, and dissociating the antibody from the antibody:protein complex, thereby obtaining antibody that specifically binds the protein. In one aspect the antibodies are selected from intact immunoglobulin molecule, a polyclonal antibody, a monoclonal antibody, a multispecific molecule, a chimeric antibody, a recombinant antibody, a humanized antibody, single chain antibodies, a Fab fragment, an F(ab′)₂ fragment, an Fv fragment, and an antibody-peptide fusion protein. The invention provides purified antibodies which bind specifically to a protein.

The invention also provides methods for using a protein to prepare and purify polyclonal and monoclonal antibodies which specifically bind the protein. The method for preparing a polyclonal antibody comprises immunizing a animal with protein under conditions to elicit an antibody response, isolating animal antibodies, attaching the protein to a substrate, contacting the substrate with isolated antibodies under conditions to allow specific binding to the protein, dissociating the antibodies from the protein, thereby obtaining purified polyclonal antibodies. The method for preparing a monoclonal antibodies comprises immunizing a animal with a protein under conditions to elicit an antibody response, isolating antibody producing cells from the animal, fusing the antibody producing cells with immortalized cells in culture to form monoclonal antibody producing hybridoma cells, culturing the hybridoma cells, and isolating monoclonal antibodies from culture.

The invention also provides a method for using an antibody to detect expression of a protein in a sample, the method comprising combining the antibody with a sample under conditions for formation of antibody:protein complexes, and detecting complex formation, wherein complex formation indicates expression of the protein in the sample. In one aspect, the sample is selected from blood, lung, lymph node, prostate, spleen, tonsil, and thymus. In a second aspect, complex formation is compared to standards and is diagnostic of a cell proliferative or inflammatory disorder.

The invention provides a method for immunopurification of a protein comprising attaching an antibody to a substrate, exposing the antibody to a sample containing the protein under conditions to allow antibody:protein complexes to form, dissociating the protein from the complex, and collecting purified protein. The invention also provides a composition comprising an antibody that specifically binds the protein and a labeling moiety or pharmaceutical agent; a kit comprising the composition; an array element comprising the antibody; and a substrate upon which the antibody is immobilized. The invention further provides a method for using a antibody to assess efficacy of a molecule or compound, the method comprising treating a sample containing protein with a molecule or compound; contacting the protein in the sample with the antibody under conditions for complex formation; determining the amount of complex formation; and comparing the amount of complex formation in the treated sample with the amount of complex formation in an untreated sample, wherein a difference in complex formation indicates efficacy of the molecule or compound.

The invention provides a method for treating a cell proliferative or inflammatory disorder comprising administering to a subject in need of therapeutic intervention a therapeutic antibody that specifically binds the protein, a multispecific molecule that specifically binds the protein, and a multispecific molecule that specifically binds the protein, or a composition comprising an antibody that specifically binds the protein and a pharmaceutical agent. The invention also provides a method for delivering a pharmaceutical or therapeutic agent to a cell comprising attaching the pharmaceutical or therapeutic agent to a multispecific molecule that specifically binds the protein and administering the multispecific molecule to a subject in need of therapeutic intervention, wherein the multispecific molecule delivers the pharmaceutical or therapeutic agent to the cell. In one aspect, the protein is active in a cell proliferative or inflammatory disorder.

The invention provides an agonist that specifically binds the protein, and a composition comprising the agonist and a pharmaceutical carrier. The invention also provides an antagonist that specifically binds the protein, and a composition comprising the antagonist and a pharmaceutical carrier. The invention further provides a pharmaceutical agent or a small drug molecule that specifically binds the protein.

The invention provides an antisense molecule of at least 18 nucleotides in length that specifically binds a portion of a polynucleotide having a nucleic acid sequence of SEQ ID NO:2 or their complements wherein the antisense molecule inhibits expression of the protein encoded by the polynucleotide. The invention also provides an antisense molecule with at least one modified internucleoside linkage or at least one nucleotide analog. The invention further provides that the modified internucleoside linkage is a phosphorothioate linkage and that the modified nucleobase is a 5-methylcytosine.

The invention provides a method for inserting a heterologous marker gene into the genomic DNA of a mammal to disrupt the expression of the endogenous polynucleotide. The invention also provides a method for using a cDNA to produce a mammalian model system, the method comprising constructing a vector containing the cDNA selected from SEQ ID NOs:2-12, transforming the vector into an embryonic stem cell, selecting a transformed embryonic stem cell, microinjecting the transformed embryonic stem cell into a mammalian blastocyst, thereby forming a chimeric blastocyst, transferring the chimeric blastocyst into-a pseudopregnant dam, wherein the dam gives birth to a chimeric offspring containing the cDNA in its germ line, and breeding the chimeric mammal to produce a homozygous, mammalian model system.

### BRIEF DESCRIPTION OF THE FIGURES

FIGS. 1A-H show the human RADCP having the amino acid sequence of SEQ ID NO:1 encoded by the Incyte cDNA having the nucleic acid sequence of SEQ ID NO:2. The translation was produced using MACDNASIS PRO software (Hitachi Software Engineering, South San Francisco Calif.).

FIGS. 2A-C show the amino acid sequence alignment between RADCP (SEQ ID NO:1), rat Maxp1 (g2459833; SEQ ID NO:13), and mouse Nore1 (g2997698; SEQ ID NO:14). The alignment was produced using the MEGALIGN program (DNASTAR, Madison Wis.).

### DESCRIPTION OF THE INVENTION

It is understood that this invention is not limited to the particular machines, materials and methods described. It is also to be understood that the terminology used herein is for the purpose of describing particular embodiments and is not intended to limit the scope of the present invention which will be limited only by the appended claims. As used herein, the singular forms “a”, “an”, and “the” include plural reference unless the context clearly dictates otherwise. For example, a reference to “a host cell” includes a plurality of such host cells known to those skilled in the art.

Unless defined otherwise, all technical and scientific terms used herein have the same meanings as commonly understood by one of ordinary skill in the art to which this invention belongs. All publications mentioned herein are cited for the purpose of describing and disclosing the cell lines, protocols, reagents and vectors which are reported in the publications and which might be used in connection with the invention. Nothing herein is to be construed as an admission that the invention is not entitled to antedate such disclosure by virtue of prior invention.

Definitions

“Antibody” refers to intact immunoglobulin molecule, a polyclonal antibody, a monoclonal antibody, a chimeric antibody, a recombinant antibody, a humanized antibody, single chain antibodies, a Fab fragment, an F(ab′)₂ fragment, an Fv fragment, and an antibody-peptide fusion protein.

“Antigenic determinant” refers to an antigenic or immunogenic epitope, structural feature, or region of an oligopeptide, peptide, or protein which is capable of inducing formation of an antibody that specifically binds the protein. Biological activity is not a prerequisite for immunogenicity.

“Array” refers to an ordered arrangement of at least two cDNAs, proteins, or antibodies on a substrate. At least one of the cDNAs, proteins, or antibodies represents a control or standard, and the other cDNA, protein, or antibody is of diagnostic or therapeutic interest. The arrangement of at least two and up to about 40,000 cDNAs, proteins, or antibodies on the substrate assures that the size and signal intensity of each labeled complex, formed between each cDNA and at least one nucleic acid, each protein and at least one ligand or antibody, or each antibody and at least one protein to which the antibody specifically binds, is individually distinguishable.

A “cancer” irefers to an adenocarcinoma, leukemia, lymphoma, melanoma, myeloma, sarcoma, teratocarcinoma, and tumors of the adrenal gland, bladder, bone, bone marrow, brain, breast, cervix, colon, esophagus, gall bladder, ganglia, heart, kidney, liver, lung, muscle, ovary, pancreas, parathyroid, penis, pituitary gland, prostate, salivary glands, skin, small intestine, spleen, stomach, testis, thymus, thyroid, and uterus.

The “complement” of a cDNA of the Sequence Listing refers to a nucleic acid molecule which is completely complementary over its full length and which will hybridize to a nucleic acid molecule under conditions of high stringency.

“cDNA” refers to an isolated polynucleotide, nucleic acid molecule, or any fragment thereof that contains from about 400 to about 12,000 nucleotides. It may have originated recombinantly or synthetically, may be double-stranded or single-stranded, may represent coding and noncoding 3′ or 5′ sequence, and generally lacks introns.

The phrase “cDNA encoding a protein” refers to a nucleic acid whose sequence closely aligns with sequences that encode conserved regions, motifs or domains identified by employing analyses well known in the art. These analyses include BLAST (Basic Local Alignment Search Tool; Altschul (1993) J Mol Evol 36:290-300; Altschul et al. (1990) J Mol Biol 215:403-410) and BLAST2 (Altschul et al. (1997) Nucleic Acids Res 25:3389-3402) which provide identity within the conserved region. Brenner et al. (1998; Proc Natl Acad Sci 95:6073-6078) who analyzed BLAST for its ability to identify structural homologs by sequence identity found 30% identity is a reliable threshold for sequence alignments of at least 150 residues and 40% is a reasonable threshold for alignments of at least 70 residues (Brenner, page 6076, column 2).

A “composition” refers to the polynucleotide and a labeling moiety; a purified protein and a pharmaceutical carrier or a heterologous, labeling or purification moiety; an antibody and a labeling moiety or pharmaceutical agent; and the like.

“Derivative” refers to a cDNA or a protein that has been subjected to a chemical modification. Derivatization of a cDNA can involve substitution of a nontraditional base such as queosine or of an analog such as hypoxanthine. These substitutions are well known in the art. Derivatization of a cDNA or a protein can also involve the replacement of a hydrogen by an acetyl, acyl, alkyl, amino, formyl, or morpholino group (for example, 5-methylcytosine). Derivative molecules retain the biological activities of the naturally occurring molecules but may confer longer lifespan or enhanced activity.

“Differential expression” refers to an increased or upregulated or a decreased or downregulated expression as detected by absence, presence, or at least two-fold change in the amount of messenger RNA or protein in a sample.

“Disorder” refers to conditions, diseases or syndromes in which the cDNAs and RADCP are differentially expressed, particularly cell proliferative and inflammatory disorders including allergies, asthma, hypereosinophilia, cancers such as prostate adenofibromatous hyperplasia, thymus hyperplasia and lung tumors.

An “expression profile” is a representation of gene expression in a sample. A nucleic acid expression profile is produced using sequencing, hybridization, or amplification (quantitative PCR) technologies and mRNAs or cDNAs from a sample. A protein expression profile, although time delayed, mirrors the nucleic acid expression profile and may use antibody or protein arrays, enzyme-linked immunosorbent assays, fluorescence-activated cell sorting, spatial immobilization such as 2D-PAGE in conjunction with a scintillation counter, mass spectrophotometry, or western analysis or affinity chromatography, to detect protein expression in a sample. The nucleic acids, proteins, or antibodies may be used in solution or attached to a substrate, and their detection is based on methods and labeling moieties well known in the art. Expression profiles may also be evaluated by methods such as electronic northern analysis, guilt-by-association, and transcript imaging. Expression profiles produced using any of the above methods may be contrasted with expression profiles produced using normal or diseased tissues. Of note is the correspondence between mRNA and protein expression has been discussed by Zweiger (2001, Transducing the Genome. McGraw-Hill, San Francisco, Calif.) and Glavas et al. (2001; T cell activation upregulates cyclic nucleotide phosphodiesterases 8A1 and 7A3, Proc Natl Acad Sci 98:6319-6342) among others.

“Fragment” refers to a chain of consecutive nucleotides from about 50 to about 5000 base pairs in length. Fragments may be used in PCR or hybridization technologies to identify related nucleic acid molecules and in binding assays to screen for a ligand. Such ligands are useful pharmaceutically to regulate replication, transcription or translation.

“Guilt-by-association” (GBA) is a method for identifying cDNAs or proteins that are associated with a specific disease, regulatory pathway, subcellular compartment, cell type, tissue type, or species by their highly significant co-expression with known markers or therapeutics.

A “hybridization complex” is formed between a cDNA and a nucleic acid of a sample when the purines of one molecule hydrogen bond with the pyrimidines of the complementary molecule, e.g., 5′-A-G-T-C-3′ base pairs with 3′-T-C-A-G-5′. Hybridization conditions, degree of complementarity and the use of nucleotide analogs affect the efficiency and stringency of hybridization reactions.

“Identity” as applied to sequences, refers to the quantification (usually percentage) of nucleotide or residue matches between at least two sequences aligned using a standardized algorithm such as Smith-Waterman alignment (Smith and Waterman (1981) J Mol Biol 147:195-197), CLUSTALW (Thompson et al. (1994) Nucleic Acids Res 22:4673-4680), or BLAST2 (Altschul (1997, supra). BLAST2 may be used in a standardized and reproducible way to insert gaps in one of the sequences in order to optimize alignment and to achieve a more meaningful comparison between them. “Similarity” uses the same algorithms but takes conservative substitution of residues into account. In proteins, similarity exceeds identity in that substitution of a valine for a leucine or isoleucine, is counted in calculating the reported percentage. Substitutions which are considered to be conservative are well known in the art.

“Isolated or “purified” refers to any molecule or compound that is separated from its natural environment and is from about 60% free to about 90% free from other components with which it is naturally associated.

“Labeling moiety” refers to any reporter molecule including radionuclides, enzymes, fluorescent, chemiluminescent, or chromogenic agents, substrates, cofactors, inhibitors, or magnetic particles than can be attached to or incorporated into a polynucleotide, protein, or antibody. Visible labels and dyes include but are not limited to anthocyanins, β glucuronidase, biotin, BIODIPY, Coomassie blue, Cy3 and Cy5, 4,6-diamidino-2-phenylindole (DAPI), digoxigenin, fluorescein, FITC, gold, green fluorescent protein, lissamine, luciferase, phycoerythrin, rhodamine, spyro red, silver, streptavidin, and the like. Radioactive markers include radioactive forms of hydrogen, iodine, phosphorous, sulfur, and the like.

“Ligand” refers to any agent, molecule, or compound which will bind specifically to a polynucleotide or to an epitope of a protein. Such ligands stabilize or modulate the activity of polynucleotides or proteins and may be composed of inorganic and/or organic substances including minerals, cofactors, nucleic acids, proteins, carbohydrates, fats, and lipids.

A “multispecific molecule” has multiple binding specificities, can bind at least two distinct epitopes or molecules, one of which may be a molecule on the surface of a cell. Antibodies can perform as or be a part of a multispecific molecule.

“Oligonucleotide” refers a single-stranded molecule from about 18 to about 60 nucleotides in length which may be used in hybridization or amplification technologies or in regulation of replication, transcription or translation. Equivalent terms are amplicon, amplimer, primer, and oligomer.

A “pharmaceutical agent” or “therapeutic agent” may be an antibody, an antisense or RNAi molecule, a multispecific molecule, a peptide, a protein, a radionuclide, a small drug molecule, a cytospecific or cytotoxic drug such as abrin, actinomyosin D, cisplatin, crotin, doxorubicin, 5-fluorouracil, methotrexate, ricin, vincristine, vinblastine, or any combination of these elements.

“Post-translational modification” of a protein can involve lipidation, glycosylation, phosphorylation, acetylation, racemization, proteolytic cleavage, and the like. These processes may occur synthetically or biochemically. Biochemical modifications will vary by cellular location, cell type, pH, enzymatic milieu, and the like.

“Probe” refers to a cDNA that hybridizes to at least one nucleic acid in a sample. Where targets are single-stranded, probes are complementary single strands. Probes can be labeled with reporter molecules for use in hybridization reactions including Southern, northern, in situ, dot blot, array, and like technologies or in screening assays.

“Protein” refers to a polypeptide or any portion thereof. A “portion” of a protein refers to that length of amino acid sequence which would retain at least one biological activity, a domain identified by PFAM or PRINTS analysis or an antigenic determinant of the protein identified using Kyte-Doolittle algorithms of the PROTEAN program (DNASTAR, Madison Wis.). An “oligopeptide” is an amino acid sequence from about five residues to about 15 residues that is used as part of a fusion protein to produce an antibody.

“RADCP” refers to a purified protein obtained from any mammalian species, including bovine, canine, murine, ovine, porcine, rodent, simian, and preferably the human species, and from any source, whether natural, synthetic, semi-synthetic, or recombinant.

“Sample” is used in its broadest sense and may comprise a bodily fluid such as ascites, blood, cerebrospinal fluid, lymph, semen, sputum, urine and the like; the soluble fraction of a cell preparation, or an aliquot of media in which cells were grown; a chromosome, an organelle, or membrane isolated or extracted from a cell; genomic DNA, RNA, or cDNA in solution or bound to a substrate; a cell; a tissue, a tissue biopsy, or a tissue print; buccal cells, skin, hair, a hair follicle; and the like.

“Specific binding” refers to a precise interaction between two molecules which is dependent upon their structure, particularly their molecular side groups. For example, the intercalation of a regulatory protein into the major groove of a DNA molecule or the binding between an epitope of a protein and an agonist, antagonist, or antibody.

“Substrate” refers to any rigid or semi-rigid support to which polynucleotides, proteins, or antibodies are bound and includes magnetic or nonmagnetic beads, capillaries or other tubing, chips, fibers, filters, gels, membranes, plates, polymers, slides, wafers, and microparticles with a variety of surface forms including channels, columns, pins, pores, trenches, and wells.

A “transcript image” (TI) is a profile of gene transcription activity in a particular tissue at a particular time. TI provides assessment of the relative abundance of expressed polynucleotides in the cDNA libraries of an EST database as described in U.S. Pat. No. 5,840,484, incorporated herein by reference.

“Variant” refers to molecules that are recognized variations of a protein or the polynucleotides that encode it. Splice variants may be determined by BLAST score, wherein the score is at least 100, and most preferably at least 400. Allelic variants have a high percent identity to the cDNAs and may differ by about three bases per hundred bases. “Single nucleotide polymorphism” (SNP) refers to a change in a single base as a result of a substitution, insertion or deletion. The change may be conservative (purine for purine) or non-conservative (purine to pyrimidine) and may or may not result in a change in an encoded amino acid or its secondary, tertiary, or quaternary structure.

The Invention

The invention is based on the discovery of RADCP, its encoding cDNAs and antibodies that specifically bind RADCP, that may be used directly or as compositions to diagnose, to stage, to treat, or to monitor the progression and/or treatment of cell proliferative and inflammatory disorders.

The present invention was discovered using a method for identifying cDNAs which coexpress with genes known to be associated with inflammation in a plurality of samples. The genes known to be involved in inflammation were CD 16, L-selectin, Src-like adapter protein (SLAP), IP-30, superoxidase homoenzyme subunits (p67phox, p47phox, and p40phox), alpha-1-antitrypsin (AAT), Clq-A, 5-lipoxygenase activating protein (FLAP), and SRC family tyrosine kinase (HCK). The literature associated with the selection of these known genes and the statistical methods for determining the probability of coexpression (p-value) are described in U.S. Ser. No. 09/195,292 incorporated by reference herein. In the table below, column 1 lists the genes that coexpressed with RADCP; column 2, the negative log of the p-value (−log p) for the coexpression of RADCP.

| Genes expressed during | |
|---|---|
| inflammation | (−log p) for coexpression with RADCP |
| CD16 | 3 |
| L-selectin | 8 |
| SLAP | 5 |
| IP-30 | 4 |
| P67phox | 6 |
| P47phox | 7 |
| P40phox | 2 |
| AAT | 2 |
| CLq-A | 0 |
| FLAP | 3 |
| HCK | 5 |

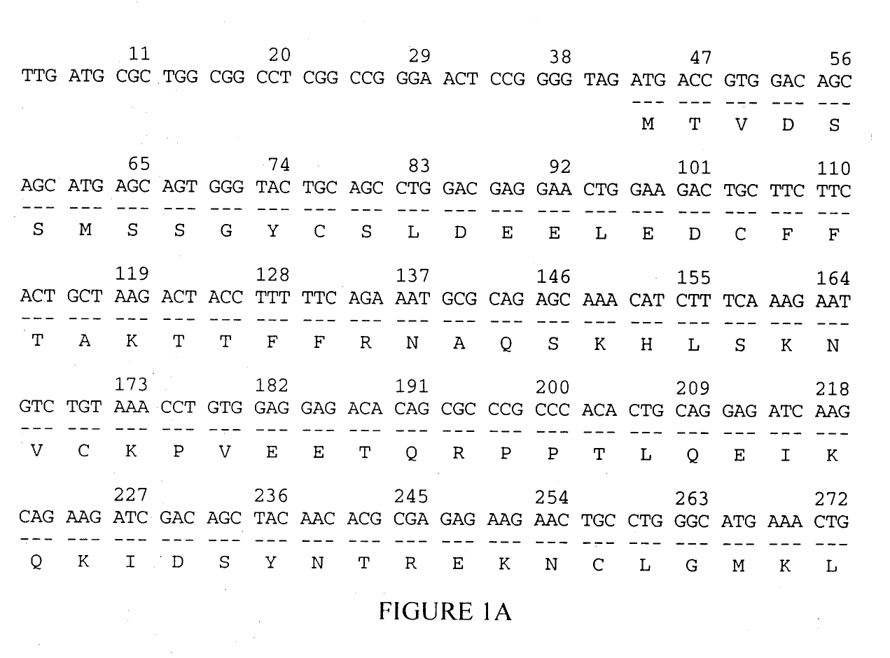

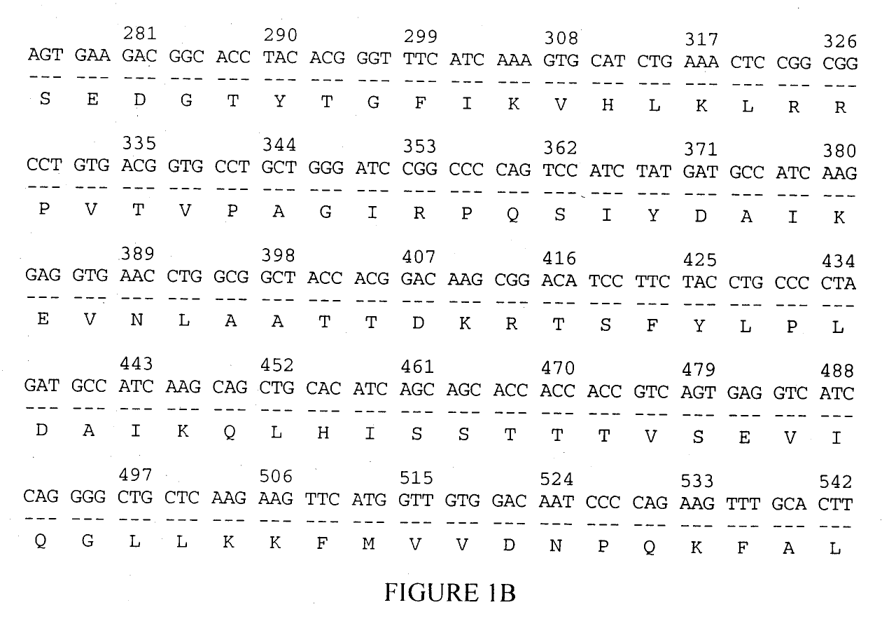

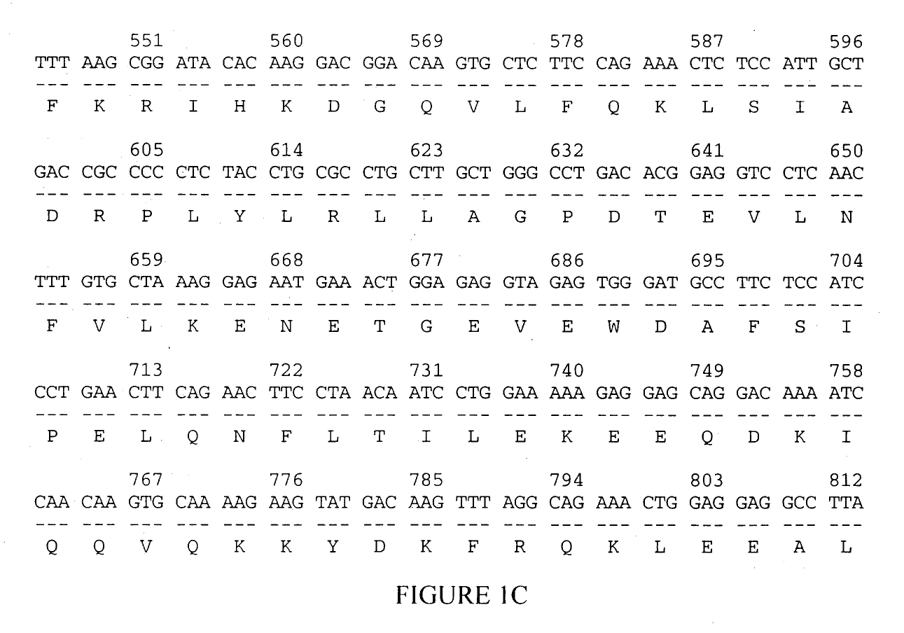

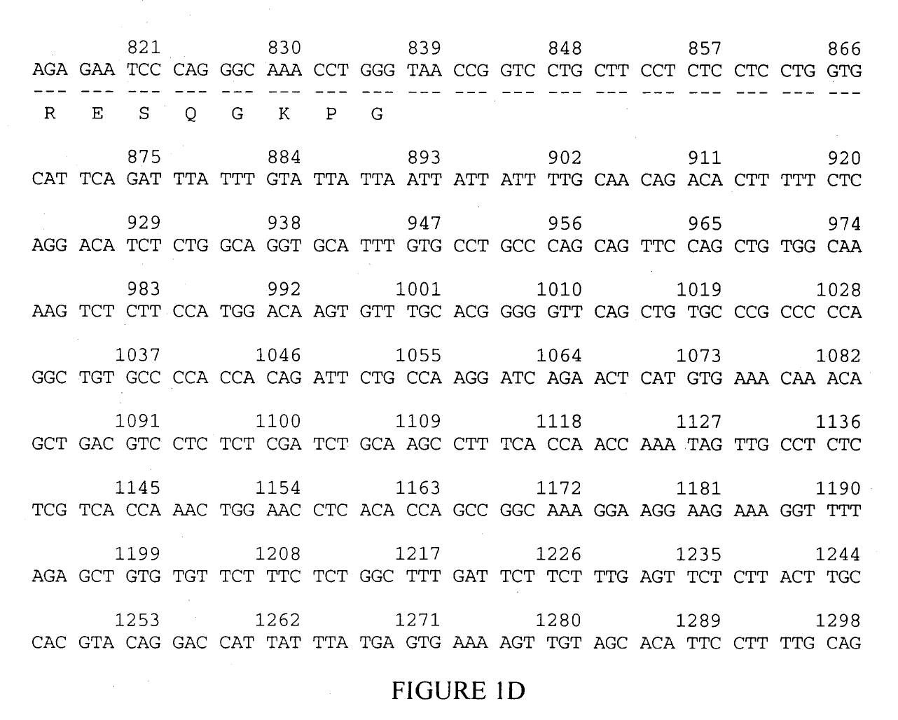

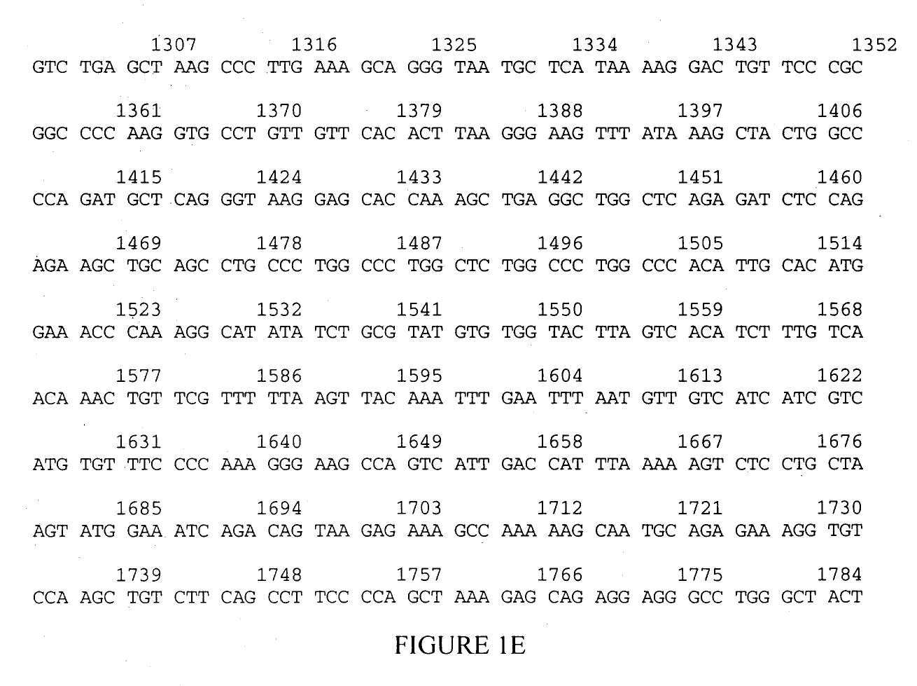

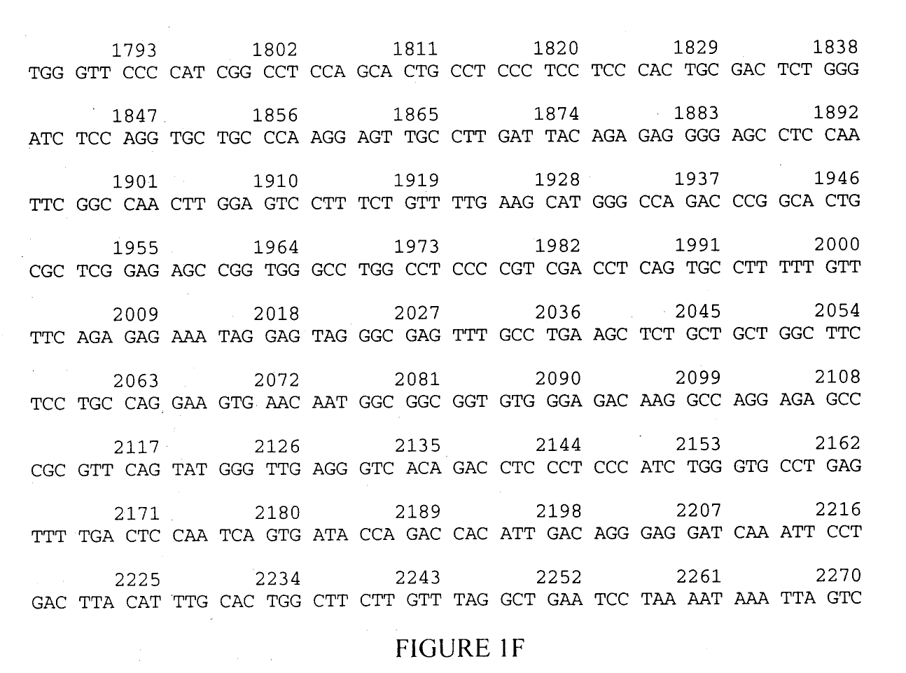

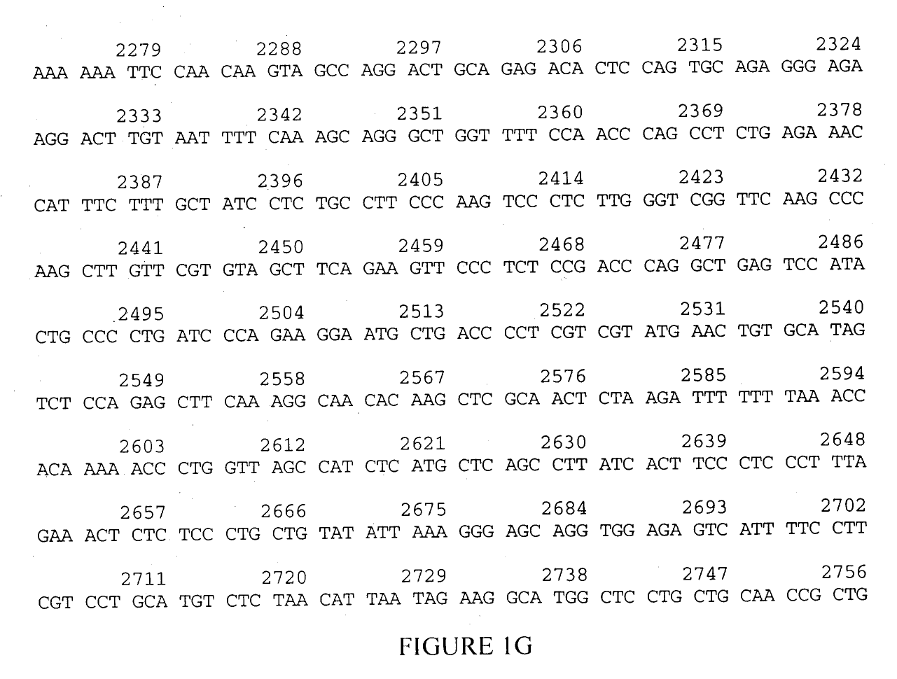

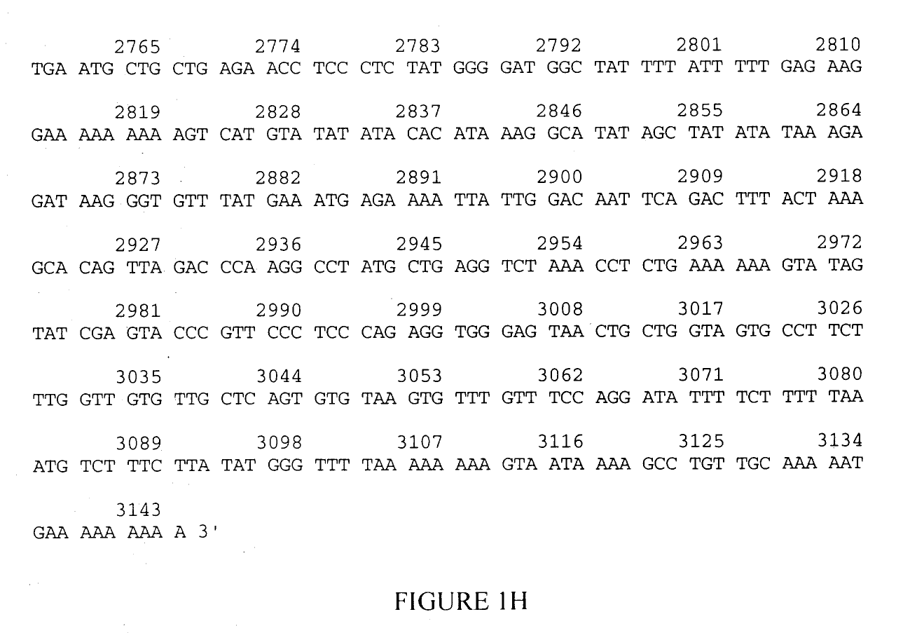

The cDNA encoding RADCP of the present invention was first identified by BLAST homology between the translation of Incyte Clone 2726173 from the ovarian tumor cDNA library (OVARTUT05), rat Maxp1 (g2459833; SEQ ID NO:13), and mouse Nore1 (g2997698; SEQ ID NO:14). The consensus sequence, SEQ ID NO:2, shown in FIGS. 1A-1H, was derived from the overlapping and/or extended cDNA sequence fragments of SEQ ID NO:3-9 presented in the table below. The sequence fragments of SEQ ID NOs:3-9 have from about 90% to about 99% identity to SEQ ID NO:2. The first column of the table shows the SEQ ID NO for the sequence fragment, the second column, the Incyte clone number; the third column, the library name; the fourth column, the nucleotide alignment, and the fifth column, percent identity between the full length cDNA and each sequence fragment.

| SEQ ID | Incyte ID | Library | Nt Alignment | % Identity |
|---|---|---|---|---|
| 3 | 2201728H1 | SPLNFET02 | 1-236 | 99 |
| 4 | 2617724F6 | OVARTUT05 | 1-162 | 99 |
| 5 | 1683926F6 | PROSNOT15 | 945-1558 | 92 |
| 6 | 1624086F6 | BRAITUT13 | 1543-2117 | 99 |
| 7 | 157250R1 | THP1PLB02 | 1653-2300 | 90 |
| 8 | 157250F1 | THP1PLB02 | 2357-3144 | 99 |
| 9 | 1208878R1 | BRSTNOT02 | 2647-3136 | 99 |

Useful fragments of the cDNA that encodes RADCP include the oligonucleotides from nucleotide 1 to nucleotide 50 of SEQ ID NO:2 or the complement thereof.

The tables in EXAMPLE VIII show the highly differential expression of the transcript encoding RADCP in the hemic and immune system, particularly in the spleen, activated eosinophils, granulocytes, and macrophages. Overexpression of RADCP in eosinophil libraries, EOSINOT02 and EOSINOT03, is associated with inflammatory disorders, particularly, allergies, asthma and hypereosinophilia. RADCP shows increased expression in eosinophils and other granulocytes in response to factors known to stimulate the immune inflammatory response. RADCP expression was increased in eosinophils activated with IL-5, a cytokine that promotes eosinophil production, and in granulocytes treated with the cytokine, GM-CSF; the mitogen, lipopolysaccharide (LPS); and the chemotactic agent, fMLP. In addition, RADCP shows overexpression in a cDNA library made from tissue of thymus diagnosed as displaying hyperplasia (THYMDIT01).

Microarray experimental results are shown in the table presented in EXAMPLE VII. RADCP is clearly differentially expressed in PBMC treated with dexamethazone±LPS as compared with matched untreated PBMC and in lung tumor compared with matched normals from the same donor. This biological data supports the use of RADCP, its encoding cDNA or an antibody that specifically binds RADCP in assays of lung tissue as diagnostics for lung cancer, particularly squamous cell carcinoma.

Mammalian variants of the cDNA encoding RADCP were identified using BLAST2 with default parameters and the LIFESEQ or ZOOSEQ databases (Incyte Genomics). The mammalian variants of RADCP, SEQ ID NOs:10-12, are listed in the table below. Column 1 lists the SEQ ID NO; column 2, the Incyte clone number; column 3, the library designation; column 4, the nucleotide (nt) alignment with SEQ ID NO:2; and column 5, the nucleotide identity compared to SEQ ID NO:2. These cDNAs are particularly useful for producing transgenic cell lines or organisms which model human disorders and upon which potential therapeutic treatments for such disorders may be tested.

| SEQ ID | | | | |
|---|---|---|---|---|
| NO | Incyte Clone | Library | Nt Homology | Nt Identity |
| 10 | 700541810H1 | RACONOT01 | 2699-2834 | 87% |
| 11 | 700632515H1 | RATHNOT02 | 42-99 | 94% |
| 12 | 700540004H1 | RACONOT01 | 2829-2864 | 97% |
| | | | 3006-3034 | 96% |
| | | | 2915-2932 | 100% |

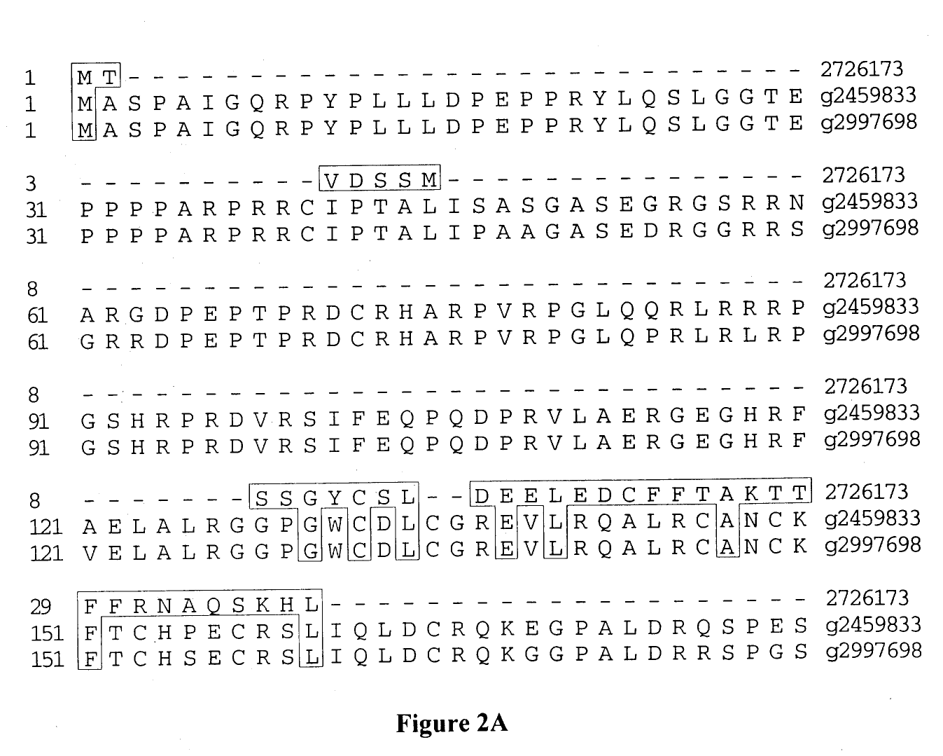

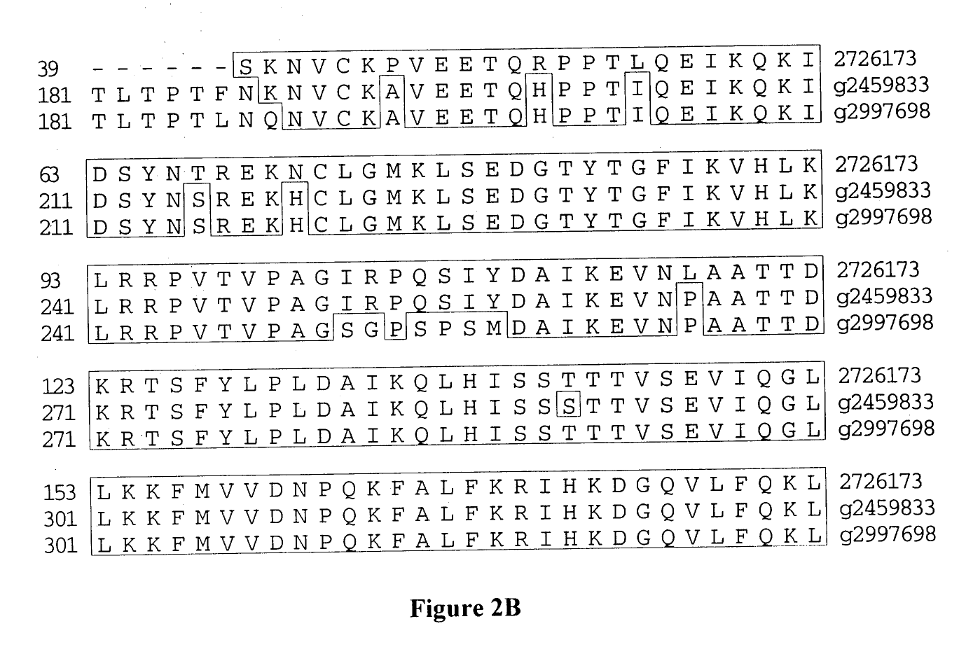

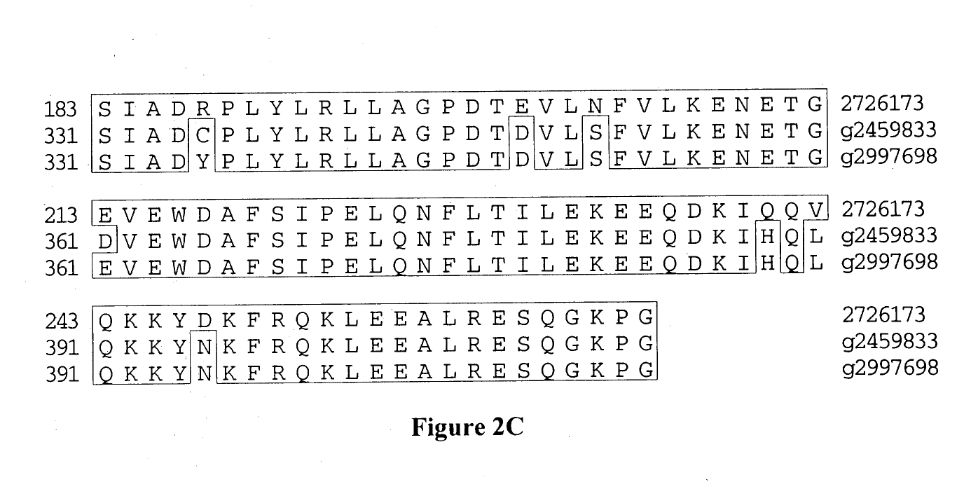

RADCP comprising the amino acid sequence of SEQ ID NO:1 is 265 amino acids in length. Pfam analysis indicates that the region of RADCP from A119 to T211 is similar to a RalGDS/AF-6 Ras association domain. This domain is found in RasGTP effectors. As shown in FIG. 2, RADCP has chemical and structural similarity with rat Maxp1 (g2459833; SEQ ID NO:13) and mouse Nore1 (g2997698; SEQ ID NO:14). In particular, RADCP and rat Maxp1 share 93% amino acid identity. RADCP and mouse Nore1 share 91% amino acid identity. The RalGDS/AF-6 Ras association domain from residue A119 to T211 in RADCP is also found in rat Maxp1 and mouse Nore1. Exemplary portions of SEQ ID NO:1 are an antigenic epitope, residue F30 to residue M75 of SEQ ID NO:1 (identified using the PROTEAN program; DNASTAR); and a biologically active portion, the conserved Ras association domain, residue A119 to residue T211 of SEQ ID NO:1.

The cDNA and fragments thereof (SEQ ID NOs:2-12) may be used in hybridization, amplification, and screening technologies to identify and distinguish among SEQ ID NO:2 and related molecules in a sample. The mammalian cDNAs may be used to produce transgenic cell lines or organisms which are model systems for human cell proliferative and inflammatory disorders, particularly allergies, asthma, hypereosinophilia, cancers such as prostate adenofibromatous hyperplasia, thymus hyperplasia and lung tumors and upon which the toxicity and efficacy of potential therapeutic treatments may be tested. Toxicology studies, clinical trials, and subject/patient treatment profiles may be performed and monitored using the cDNAs, proteins, antibodies and molecules and compounds identified using the cDNAs and proteins of the present invention.

It will be appreciated by those skilled in the art that as a result of the degeneracy of the genetic code, a multitude of cDNAs encoding RADCP, some bearing minimal similarity to the cDNAs of any known and naturally occurring gene, may be produced. Thus, the invention contemplates each and every possible variation of cDNA that could be made by selecting combinations based on possible codon choices. These combinations are made in accordance with the standard triplet genetic code as applied to the polynucleotides encoding naturally occurring RADCP, and all such variations are to be considered as being specifically disclosed.

Characterization and use of the Invention

cDNA Libraries

In a particular embodiment disclosed herein, mRNA is isolated from mammalian cells and tissues using methods which are well known to those skilled in the art and used to prepare the cDNA libraries. The Incyte cDNAs were isolated from mammalian cDNA libraries prepared as described in the EXAMPLES I-III. The consensus sequence is present in a single clone insert, or chemically assembled, based on the electronic assembly from sequenced fragments including Incyte cDNAs and extension and/or shotgun sequences. Computer programs, such as PHRAP (Green, supra) and the AUTOASSEMBLER application (ABI), are used in sequence assembly and are described in EXAMPLE V. After verification of the 5′ and 3′ sequence, at least one representative cDNA which encodes RADCP is designated a reagent for research and development.

Sequencing

Methods for sequencing nucleic acids are well known in the art and may be used to practice any of the embodiments of the invention. These methods employ enzymes such as the Klenow fragment of DNA polymerase I, SEQUENASE, Taq DNA polymerase and thermostable T7 DNA polymerase (Amersham Biosciences (APB), Piscataway N.J.), or combinations of polymerases and proofreading exonucleases (Invitrogen, Carlsbad Calif.). Sequence preparation is automated with machines such as the MICROLAB 2200 system (Hamilton, Reno Nev.) and the DNA ENGINE thermal cycler (MJ Research, Watertown Mass.) and sequencing, with the PRISM 3700, 377 or 373 DNA sequencing systems (ABI) or the MEGABACE 1000 DNA sequencing system (APB).

After sequencing, sequence fragments are assembled to obtain and verify the sequence of the full length cDNA. The full length sequence usually resides in a single clone insert which may contain up to 5000 bases. Since sequencing reactions generally reveal no more than 700 bases per reaction, it is more often than not necessary to carry out several sequencing reactions, and procedures such as shotgun sequencing or PCR extension, in order to obtain the full length sequence.

Shotgun sequencing involves randomly breaking the original insert into segments of various sizes and cloning these fragments into vectors. The fragments are sequenced and reassembled using overlapping ends until the entire sequence of the original insert is known. Shotgun sequencing methods are well known in the art and use thermostable DNA polymerases, heat-labile DNA polymerases, and primers chosen from representative regions flanking the cDNAs of interest. Incomplete assembled sequences are inspected for identity using various algorithms or programs such as CONSED (Gordon (1998) Genome Res 8:195-202) which are well known in the art.

PCR-based methods may be used to extend the sequences of the invention. PCR extension is described in EXAMPLE IV.

The nucleic acid sequences of the cDNAs presented in the Sequence Listing were prepared by automated methods and may contain occasional sequencing errors and unidentified nucleotides, designated with an N, that reflect state-of-the-art technology at the time the cDNA was sequenced. Vector, linker, and polyA sequences were masked using algorithms and programs based on BLAST, dynamic programming, and dinucleotide nearest neighbor analysis. Ns and SNPs can be verified either by resequencing the cDNA or using algorithms to compare multiple sequences that overlap the area in which the Ns or SNP occur. Both of these techniques are well known to and used by those skilled in the art. The sequences may be analyzed using a variety of algorithms described in Ausubel et al. (1997; Short Protocols in Molecular Biology, John Wiley & Sons, New York N.Y., unit 7.7) and in Meyers (1995; Molecular Biology and Biotechnology, Wiley VCH, New York N.Y., pp. 856-853).

Hybridization

The cDNA and fragments thereof can be used in hybridization technologies for various purposes. A probe may be designed or derived from unique regions such as the 5′ regulatory region or from a nonconserved region (i.e., 5′ or 3′ of the nucleotides encoding the conserved catalytic domain of the protein) and used in protocols to identify naturally occurring molecules encoding RADCP, allelic variants, or related molecules. The probe may be DNA or RNA, may be single-stranded, and should have at least 50% sequence identity to any of the nucleic acid sequences, SEQ ID NOs:2-12. Hybridization probes may be produced using oligolabeling, nick-translation, end-labeling, or PCR amplification in the presence of a reporter molecule. A vector containing the cDNA or a fragment thereof may be used to produce an mRNA probe in vitro by addition of an RNA polymerase and labeled nucleotides. These procedures may be conducted using kits such as those provided by APB.

The stringency of hybridization is determined by G+C content of the probe, salt concentration, and temperature. In particular, stringency can be increased by reducing the concentration of salt or raising the hybridization temperature. Hybridization techniques are well known in the art, have been described in Example VII, and are reviewed in Ausubel (supra) and Sambrook et al. (1989) Molecular Cloning, A Laboratory Manual, Cold Spring Harbor Press, Plainview N.Y.

Arrays may be prepared and analyzed using methods well known in the art. Oligonucleotides or cDNAs may be used as hybridization probes or targets to monitor the expression level of large numbers of genes simultaneously or to identify genetic variants, mutations, and single nucleotide polymorphisms. Arrays may be used to determine gene function; to understand the genetic basis of a condition, disease, or disorder; to diagnose a condition, disease, or disorder; and to develop and monitor the activities of therapeutic agents. See, e.g., U.S. Pat. No. 5,474,796; Schena et al. (1996) Proc Natl Acad Sci 93:10614-10619; Heller et al. (1997) Proc Natl Acad Sci 94:2150-2155; U.S. Pat. No. 5,605,662.

Hybridization probes are also useful in mapping the naturally occurring genomic sequence. The probes may be hybridized to a particular chromosome, a specific region of a chromosome, or an artificial chromosome construction. Such constructions include human artificial chromosomes, yeast artificial chromosomes, bacterial artificial chromosomes, bacterial P1 constructions, or the cDNAs of libraries made from single chromosomes.

QPCR

QPCR is a method for quantifying a nucleic acid molecule based on detection of a fluorescent signal produced during PCR amplification (Gibson et al. (1996) Genome Res 6:995-1001; Heid et al. (1996) Genome Res 6:986-994). Amplification is carried out on machines such as the PRISM 7700 detection system (ABI) which consists of a 96-well thermal cycler connected to a laser and charge-coupled device (CCD) optics system. To perform QPCR, a PCR reaction is carried out in the presence of a doubly labeled probe. The probe, which is designed to anneal between the standard forward and reverse PCR primers, is labeled at the 5′ end by a fluorogenic reporter dye such as 6-carboxyfluorescein (6-FAM) and at the 3′ end by a quencher molecule such as 6-carboxy-tetramethyl-rhodamine (TAMRA). As long as the probe is intact, the 3′ quencher extinguishes fluorescence by the 5′ reporter. However, during each primer extension cycle, the annealed probe is degraded as a result of the intrinsic 5′ to 3′ nuclease activity of Taq polymerase (Holland et al. (1991) Proc Natl Acad Sci 88:7276-7280). This degradation separates the reporter from the quencher, and fluorescence is detected every few seconds by the CCD. The higher the starting copy number of the nucleic acid, the sooner an increase in fluorescence is observed. A cycle threshold (C_{T}) value, representing the cycle number at which the PCR product crosses a fixed threshold of detection is determined by the instrument software. The C_{T} is inversely proportional to the copy number of the template and can therefore be used to calculate either the relative or absolute initial concentration of the nucleic acid molecule in the sample. The relative concentration of two different molecules can be calculated by determining their respective C_{T} values (comparative C_{T} method). Alternatively, the absolute concentration of the nucleic acid molecule can be calculated by constructing a standard curve using a housekeeping molecule of known concentration. The process of calculating C_{T} values, preparing a standard curve, and determining starting copy number is performed using SEQUENCE DETECTOR 1.7 software (ABI).

Expression

Any one of a multitude of cDNAs encoding RADCP may be cloned into a vector and used to express the protein, or portions thereof, in host cells. The nucleic acid sequence can be engineered by such methods as DNA shuffling (U.S. Pat. No. 5,830,721) and site-directed mutagenesis to create new restriction sites, alter glycosylation patterns, change codon preference to increase expression in a particular host, produce splice variants, extend half-life, and the like. The expression vector may contain transcriptional and translational control elements (promoters, enhancers, specific initiation signals, and polyadenylated 3′ sequence) from various sources which have been selected for their efficiency in a particular host. The vector, cDNA, and regulatory elements are combined using in vitro recombinant DNA techniques, synthetic techniques, and/or in vivo genetic recombination techniques well known in the art and described in Sambrook (supra, ch. 4, 8, 16 and 17).

A variety of host systems may be transformed with an expression vector. These include, but are not limited to, bacteria transformed with recombinant bacteriophage, plasmid, or cosmid DNA expression vectors; yeast transformed with yeast expression vectors; insect cell systems transformed with baculovirus expression vectors or plant cell systems transformed with expression vectors containing viral and/or bacterial elements (Ausubel supra, unit 16). In mammalian cell systems, an adenovirus transcriptional/translational complex may be utilized. After sequences are ligated into the E1 or E3 region of the viral genome, the infective virus is used to transform and express the protein in host cells. The Rous sarcoma virus enhancer or SV40 or EBV-based vectors may also be used for high-level protein expression.

Routine cloning, subcloning, and propagation of nucleic acid sequences can be achieved using the multifunctional pBLUESCRIPT vector (Stratagene, La Jolla Calif.) or pSPORT1 plasmid (Invitrogen). Introduction of a nucleic acid sequence into the multiple cloning site of these vectors disrupts the lacZ gene and allows colorimetric screening for transformed bacteria. In addition, these vectors may be useful for in vitro transcription, dideoxy sequencing, single strand rescue with helper phage, and creation of nested deletions in the cloned sequence.

For long term production of recombinant proteins, the vector can be stably transformed into cell lines along with a selectable or visible marker gene on the same or on a separate vector. After transformation, cells are allowed to grow for about 1 to 2 days in enriched media and then are transferred to selective media. Selectable markers, antimetabolite, antibiotic, or herbicide resistance genes, confer resistance to the relevant selective agent and allow growth and recovery of cells which successfully express the introduced sequences. Resistant clones identified either by survival on selective media or by the expression of visible markers may be propagated using culture techniques. Visible markers are also used to estimate the amount of protein expressed by the introduced genes. Verification that the host cell contains the desired cDNA is based on DNA-DNA or DNA-RNA hybridizations or PCR amplification.

The host cell may be chosen for its ability to modify a recombinant protein in a desired fashion. Such modifications include acetylation, carboxylation, glycosylation, phosphorylation, lipidation, acylation and the like. Post-translational processing which cleaves a “prepro” form may also be used to specify protein targeting, folding, and/or activity. Different host cells which have specific cellular machinery and characteristic mechanisms for post-translational activities may be chosen to ensure the correct modification and processing of the recombinant protein.

Recovery of Proteins from Cell Culture

Heterologous moieties engineered into a vector for ease of purification include glutathione S-transferase (GST), 6×His, FLAG, MYC, and the like. GST and 6-His are purified using affinity matrices such as immobilized glutathione and metal-chelate resins, respectively. FLAG and MYC are purified using monoclonal and polyclonal antibodies. For ease of separation following purification, a sequence encoding a proteolytic cleavage site may be part of the vector located between the protein and the heterologous moiety. Methods for recombinant protein expression and purification are discussed in Ausubel (supra, unit 16).

Protein Identification

Several techniques have been developed which permit rapid identification of proteins using high performance liquid chromatography (HPLC) and mass spectrometry (MS). Beginning with a sample containing proteins, the method is: 1) proteins are separated using electrophoresis, 2) selected proteins are excised from the gel and digested with a protease to produce a set of peptides; and 3) the peptides are subjected to HPLC to analyze amino acid content or MS to derive peptide ion mass and spectral pattern information. The MS information is used to identify the protein by comparing it with information in a protein database (Shevenko et al. (1996) Proc Natl Acad Sci 93:14440-14445).

Proteins are separated using isoelectric focusing (IEF) in the first dimension followed by SDS-PAGE in the second dimension. For IEF, an immobilized pH gradient strip is useful to increase reproducibility and resolution of the separation. Alternative techniques may be used to improve resolution of very basic, hydrophobic, or high molecular weight proteins. The separated proteins are detected using a stain or dye such as silver stain, Coomassie blue, or spyro red (Molecular Probes, Eugene Oreg.) that is compatible with MS. Gels may be blotted onto a PVDF membrane for western analysis and optically scanned using a STORM scanner (APB) to produce a computer-readable output which is analyzed by pattern recognition software such as MELANIE (GeneBio, Geneva, Switzerland). The software annotates individual spots by assigning a unique identifier and calculating their respective x,y coordinates, molecular masses, isoelectric points, and signal intensity. Individual spots of interest, such as those representing differentially expressed proteins, are excised and proteolytically digested with a site-specific protease such as trypsin or chymotrypsin, singly or in combination, to generate a set of small peptides, preferably in the range of 1-2 kDa. Prior to digestion, samples may be treated with reducing and alkylating agents, and following digestion, the peptides are then separated by liquid chromatography or capillary electrophoresis and analyzed using MS.

MS converts components of a sample into gaseous ions, separates the ions based on their mass-to-charge ratio, and determines relative abundance. For peptide mass fingerprinting analysis, a MALDI-TOF (Matrix Assisted Laser Desorption/Ionization-Time of Flight), ESI (Electrospray Ionization), and TOF-TOF (Time of Flight/Time of Flight) machines are used to determine a set of highly accurate peptide masses. Using analytical programs, such as TURBOSEQUEST software (Finnigan, San Jose Calif.), the MS data is compared against a database of theoretical MS data derived from known or predicted proteins. A minimum match of three peptide masses is used for reliable protein identification. If additional information is needed for identification, Tandem-MS may be used to derive information about individual peptides. In tandem-MS, a first stage of MS is performed to determine individual peptide masses. Then selected peptide ions are subjected to fragmentation using a technique such as collision induced dissociation (CID) to produce an ion series. The resulting fragmentation ions are analyzed in a second round of MS, and their spectral pattern may be used to determine a short stretch of amino acid sequence (Dancik et al. (1999) J Comput Biol 6:327-342). Assuming the protein is represented in the database, a combination of peptide mass and fragmentation data, together with the calculated MW and pI of the protein, will usually yield an unambiguous identification. If no match is found, protein sequence can be obtained using direct chemical sequencing procedures well known in the art (cf. Creighton (1984) Proteins, Structures and Molecular Properties, W H Freeman, New York N.Y.).

Chemical Synthesis of Peptides

Proteins or portions thereof may be produced not only by recombinant methods, but also by using chemical methods well known in the art. Solid phase peptide synthesis may be carried out in a batchwise or continuous flow process which sequentially adds α-amino- and side chain-protected amino acid residues to an insoluble polymeric support via a linker group. A linker group such as methylamine-derivatized polyethylene glycol is attached to poly(styrene-co-divinylbenzene) to form the support resin. The amino acid residues are N-α-protected by acid labile Boc (t-butyloxycarbonyl) or base-labile Fmoc (9-fluorenylmethoxycarbonyl). The carboxyl group of the protected amino acid is coupled to the amine of the linker group to anchor the residue to the solid phase support resin. Trifluoroacetic acid or piperidine are used to remove the protecting group in the case of Boc or Fmoc, respectively. Each additional amino acid is added to the anchored residue using a coupling agent or pre-activated amino acid derivative, and the resin is washed. The full length peptide is synthesized by sequential deprotection, coupling of derivitized amino acids, and washing with dichloromethane and/or N,N-dimethylformamide. The peptide is cleaved between the peptide carboxy terminus and the linker group to yield a peptide acid or amide. (Novabiochem 1997/98 Catalog and Peptide Synthesis Handbook, San Diego Calif. pp. S1-S20). Automated synthesis may also be carried out on machines such as the 431A peptide synthesizer (ABI). A protein or portion thereof may be purified by preparative HPLC and its composition confirmed by amino acid analysis or by sequencing (Creighton, supra)

Antibodies

Antibodies, or immunoglobulins (Ig), are components of immune response expressed on the surface of or secreted into the circulation by B cells. The prototypical antibody is a tetramer composed of two identical heavy polypeptide chains (H-chains) and two identical light polypeptide chains (L-chains) interlinked by disulfide bonds which binds and neutralizes foreign antigens. Based on their H-chain, antibodies are classified as IgA, IgD, IgE, IgG or IgM. The most common class, IgG, is tetrameric while other classes are variants or multimers of the basic structure.

Antibodies are described in terms of their two functional domains. Antigen recognition is mediated by the Fab (antigen binding fragment) region of the antibody, while effector functions are mediated by the Fe (crystallizable fragment) region. The binding of antibody to antigen triggers destruction of the antigen by phagocytic white blood cells such as macrophages and neutrophils. These cells express surface Fe receptors that specifically bind to the Fe region of the antibody and allow the phagocytic cells to destroy antibody-bound antigen. Fe receptors are single-pass transmembrane glycoproteins containing about 350 amino acids whose extracellular portion typically contains two or three Ig domains (Sears et al. (1990) J Immunol 144:371-378).

Preparation and Screening of Antibodies

Various hosts including mice, rats, rabbits, goats, llamas, camels, and human cell lines may be immunized by injection with an antigenic determinant. Adjuvants such as Freund's, mineral gels, and surface active substances such as lysolecithin, pluronic polyols, polyanions, peptides, oil emulsions, keyhole limpet hemacyanin (KLH; Sigma-Aldrich, St Louis Mo.), and dinitrophenol may be used to increase immunological response. In humans, BCG (bacilli Calmette-Guerin) and Corynebacterium parvum increase response. The antigenic determinant may be an oligopeptide, peptide, or protein. When the amount of antigenic determinant allows immunization to be repeated, specific polyclonal antibody with high affinity can be obtained (Klinman and Press (1975) Transplant Rev 24:41-83). Oligopepetides which may contain between about five and about fifteen amino acids identical to a portion of the endogenous protein may be fused with proteins such as KLH in order to produce antibodies to the chimeric molecule.

Monoclonal antibodies may be prepared using any technique which provides for the production of antibodies by continuous cell lines in culture. These include the hybridoma technique, the human B-cell hybridoma technique, and the EBV-hybridoma technique (Kohler et al. (1975) Nature 256:495-497; Kozbor et al. (1985) J Immunol Methods 81:31-42; Cote et al. (1983) Proc Natl Acad Sci 80:2026-2030; and Cole et al. (1984) Mol Cell Biol 62:109-120).

Chimeric antibodies may be produced by techniques such as splicing of mouse antibody genes to human antibody genes to obtain a molecule with appropriate antigen specificity and biological activity (Morrison et al. (1984) Proc Natl Acad Sci 81:6851-6855; Neuberger et al. (1984) Nature 312:604-608; and Takeda et al. (1985) Nature 314:452-454). Alternatively, techniques described for antibody production may be adapted, using methods known in the art, to produce specific, single chain antibodies. Antibodies with related specificity, but of distinct idiotypic composition, may be generated by chain shuffling from random combinatorial immunoglobulin libraries (Burton (1991) Proc Natl Acad Sci 88:10134-10137). Antibody fragments which contain specific binding sites for an antigenic determinant may also be produced. For example, such fragments include, but are not limited to, F(ab′)₂ fragments produced by pepsin digestion of the antibody molecule and Fab fragments generated by reducing the disulfide bridges of the F(ab)₂ fragments. Alternatively, Fab expression libraries may be constructed to allow rapid and easy identification of monoclonal Fab fragments with the desired specificity (Huse et al. (1989) Science 246:1275-1281).

Antibodies may also be produced by inducing production in the lymphocyte population or by screening immunoglobulin libraries or panels of highly specific binding reagents as disclosed in Orlandi et al. (1989; Proc Natl Acad Sci 86:3833-3837) or Winter et al. (1991; Nature 349:293-299). A protein may be used in screening assays of phagemid or B-lymphocyte immunoglobulin libraries to identify antibodies having a desired specificity. Numerous protocols for competitive binding or immunoassays using either polyclonal or monoclonal antibodies with established specificities are well known in the art.

Antibody Specificity

Various methods such as Scatchard analysis combined with radioimmunoassay techniques may be used to assess the affinity of particular antibodies for a protein. Affinity is expressed as an association constant, Kₐ, which is defined as the molar concentration of protein-antibody complex divided by the molar concentrations of free antigen and free antibody under equilibrium conditions. The Kₐ determined for a preparation of polyclonal antibodies, which are heterogeneous in their affinities for multiple antigenic determinants, represents the average affinity, or avidity, of the antibodies. The Kₐ determined for a preparation of monoclonal antibodies, which are specific for a particular antigenic determinant, represents a true measure of affinity. High-affinity antibody preparations with Kₐ ranging from about 10⁹ to 10¹² L/mole are commonly used in immunoassays in which the protein-antibody complex must withstand rigorous manipulations. Low-affinity antibody preparations with Kₐ ranging from about 10⁶ to 10⁷ L/mole are preferred for use in immunopurification and similar procedures which ultimately require dissociation of the protein, preferably in active form, from the antibody (Catty (1988) Antibodies, Volume I: A Practical Approach, IRL Press, Washington D.C.; Liddell and Cryer (1991) A Practical Guide to Monoclonal Antibodies, John Wiley & Sons, New York N.Y.).

The titer and avidity of polyclonal antibody preparations may be further evaluated to determine the quality and suitability of such preparations for certain downstream applications. For example, a polyclonal antibody preparation containing about 5-10 mg specific antibody/ml, is generally employed in procedures requiring precipitation of protein-antibody complexes. Procedures for making antibodies, evaluating antibody specificity, titer, and avidity, and guidelines for antibody quality and usage in various applications, are discussed in Catty (supra) and Ausubel (supra) pp. 11.1-11.31.

Diagnostics

Differential expression of RADCP or their encoding mRNAs and at least one of the assays below can be used to diagnose cell proliferative and inflammatory disorders, particularly allergies, asthma, hypereosinophilia, cancers such as prostate adenofibromatous hyperplasia, thymus hyperplasia and lung tumors or to monitor mRNA or protein levels during therapeutic intervention. Similarly antibodies which specifically bind RADCP may be used to quantitate the protein and to diagnose cell proliferative and inflammatory disorders, particularly allergies, asthma, hypereosinophilia, cancers such as prostate adenofibromatous hyperplasia, thymus hyperplasia and lung tumors.

Labeling of Molecules for Assay

A wide variety of reporter molecules and conjugation techniques are known by those skilled in the art and may be used in various nucleic acid, amino acid, and antibody assays. Synthesis of labeled molecules may be achieved using kits such as those supplied by Promega (Madison Wis.) or APB for incorporation of a labeled nucleotide such as ³²P-dCTP (APB), Cy3-dCTP or Cy5-dCTP (Qiagen-Operon, Alameda Calif.), or amino acid such as ³⁵S-methionine (APB). Nucleotides and amino acids may be directly labeled with a variety of substances including fluorescent, chemiluminescent, or chromogenic agents, and the like, by chemical conjugation to amines, thiols and other groups present in the molecules using reagents such as BIODIPY or FITC (Molecular Probes).

Nucleic Acid Assays

The cDNAs, fragments, oligonucleotides, complementary RNAs, and peptide nucleic acids (PNA) may be used to detect and quantify differential gene expression for diagnosis of a disorder. Similarly antibodies which specifically bind the protein may be used to quantitate the protein. Cell proliferative disorders are associated with such differential expression. The diagnostic assay may use hybridization or amplification technology to compare gene expression in a biological sample from a patient to standard samples in order to detect differential gene expression. Qualitative or quantitative methods for this comparison are well known in the art.

Expression Profiles

An expression profile comprises the expression of a plurality of cDNAs or protein as measured using standard assays with a sample. The cDNAs, proteins or antibodies of the invention may be used as elements on a array to produce an expression profile. In one embodiment, the array is used to diagnose or monitor the progression of disease.

For example, the cDNA or probe may be labeled by standard methods and added to a biological sample from a patient under conditions for the formation of hybridization complexes. After an incubation period, the sample is washed and the amount of label (or signal) associated with hybridization complexes, is quantified and compared with a standard value. If complex formation in the patient sample is altered in comparison to either a normal or disease standard, then differential expression indicates the presence of a disorder.

In order to provide standards for establishing differential expression, normal and disease expression profiles are established. This is accomplished by combining a sample taken from a normal subject, either animal or human, with a cDNA under conditions for hybridization to occur. Standard hybridization complexes may be quantified by comparing the values obtained using normal subjects with values from an experiment in which a known amount of a purified, control sequence is used. Standard values obtained in this manner may be compared with values obtained from samples from patients who were diagnosed with a particular condition, disease, or disorder. Deviation from standard values toward those associated with a particular disorder is used to diagnose or stage that disorder.

By analyzing changes in patterns of gene expression, disease can be diagnosed at earlier stages before the patient is symptomatic. The invention can be used to formulate a prognosis and to design a treatment regimen. The invention can also be used to monitor the efficacy of treatment. For treatments with known side effects, the array is employed to improve the treatment regimen. A dosage is established that causes a change in genetic expression patterns indicative of successful treatment. Expression patterns associated with the onset of undesirable side effects are avoided. This approach may be more sensitive and rapid than waiting for the patient to show inadequate improvement, or to manifest side effects, before altering the course of treatment.

In another embodiment, animal models which mimic a human disease can be used to characterize expression profiles associated with a particular condition, disease, or disorder; or treatment of the condition, disease, or disorder. Novel treatment regimens may be tested in these animal models using arrays to establish and then follow expression profiles over time. In addition, arrays may be used with cell cultures or tissues removed from animal models to rapidly screen large numbers of candidate drug molecules, looking for ones that produce an expression profile similar to those of known therapeutic drugs, with the expectation that molecules with the same expression profile will likely have similar therapeutic effects. Thus, the invention provides the means to rapidly determine the molecular mode of action of a drug.

Such assays may also be used to evaluate the efficacy of a particular therapeutic treatment regimen in animal studies or in clinical trials or to monitor the treatment of an individual patient. Once the presence of a condition is established and a treatment protocol is initiated, diagnostic assays may be repeated on a regular basis to determine if the level of expression in the patient begins to approximate that which is observed in a normal subject. The results obtained from successive assays may be used to show the efficacy of treatment over a period ranging from several days to years.

Protein Assays

Immunological methods for detecting and measuring complex formation as a measure of protein expression using either specific polyclonal or monoclonal antibodies are known in the art. Examples of such techniques include antibody or protein arrays, ELISA, FACS, spatial immobilization such as 2D-PAGE and SC, HPLC or MS, RIAs and western analysis. Such immunoassays typically involve the measurement of complex formation between the protein and its specific antibody. These assays and their quantitation against purified, labeled standards are well known in the art (Ausubel, supra, unit 10.1-10.6). A two-site, monoclonal-based immunoassay utilizing antibodies reactive to two non-interfering epitopes is preferred, but a competitive binding assay may be employed (Pound (1998) Immunochemical Protocols, Humana Press, Totowa N.J.).

These methods are also useful for diagnosing diseases that show differential protein expression. Normal or standard values for protein expression are established by combining body fluids or cell extracts taken from a normal mammalian or human subject with specific antibodies to a protein under conditions for complex formation. Standard values for complex formation in normal and diseased tissues are established by various methods, often photometric means. Then complex formation as it is expressed in a subject sample is compared with the standard values. Deviation from the normal standard and toward the diseased standard provides parameters for disease diagnosis or prognosis while deviation away from the diseased and toward the normal standard may be used to evaluate treatment efficacy.

Recently, antibody arrays have allowed the development of techniques for high-throughput screening of recombinant antibodies. Such methods use robots to pick and grid bacteria containing antibody genes, and a filter-based ELISA to screen and identify clones that express antibody fragments. Because liquid handling is eliminated and the clones are arrayed from master stocks, the same antibodies can be spotted multiple times and screened against multiple antigens simultaneously. Antibody arrays are highly useful in the identification of differentially expressed proteins. See de Wildt et al. (2000) Nature Biotechnol 18:989-94.

Therapeutics

Chemical and structural similarity exists between regions of RADCP (SEQ ID NO:1), rat Maxp1 (g2459833; SEQ ID NO:13), and mouse Nore1 (g2997698; SEQ ID NO:14); all of which have RalGDS/AF-6 Ras association domains as shown in FIG. 2. RADCP appears to play a role in cell proliferative and inflammatory disorders, particularly allergies, asthma, hypereosinophilia, cancers such as prostate adenofibromatous hyperplasia, thymus hyperplasia and lung tumors.

Differential expression of RADCP as associated with inflammation was described above and in U.S. Ser. No. 09/349,015. RADCP clearly plays a role in allergies, asthma, hypereosinophilia, cancers such as prostate adenofibromatous hyperplasia, thymus hyperplasia and lung tumors.

In the treatment of asthma, hypereosinophilia, and thymus hyperplasia, disorders associated with increased expression of the protein, it is desirable to decrease expression or protein activity. In one embodiment, a pharmaceutical agent such as an inhibitor, antagonist or antibody that specifically binds the protein may be administered to a subject to treat a condition associated with increased expression or activity. In another embodiment, a pharmaceutical composition comprising an inhibitor, antagonist in conjunction with a pharmaceutical carrier may be administered to a subject to treat a condition associated with the increased expression or activity of the endogenous protein. In one aspect, an antibody that specifically binds RADCP acts as a carrier to effect delivery of a pharmaceutical agent. In an additional embodiment, a vector expressing the complement of the cDNA or fragments thereof may be administered to a subject to treat the disorder. Any of the cDNAs, complementary molecules, or fragments thereof, proteins or portions thereof, vectors delivering these nucleic acid molecules or expressing the proteins, therapeutic antibodies, and ligands binding the cDNA or protein may be administered in combination with other therapeutic agents. Selection of the agents for use in combination therapy may be made by one of ordinary skill in the art according to conventional pharmaceutical principles. A combination of therapeutic agents may act synergistically to affect treatment of a particular disorder at a lower dosage of each agent.

Modification of Gene Expression Using Nucleic Acids

Gene expression may be modified by designing complementary or antisense molecules (DNA, RNA, or PNA) to the control, 5′, 3′, or other regulatory regions of the gene encoding RADCP. Oligonucleotides designed to inhibit transcription initiation are preferred. Similarly, inhibition can be achieved using triple helix base-pairing which inhibits the binding of polymerases, transcription factors, or regulatory molecules (Gee et al. In: Huber and Carr (1994) Molecular and Immunologic Approaches, Futura Publishing, Mt. Kisco N.Y., pp. 163-177). A complementary molecule may also be designed to block translation by preventing binding between ribosomes and mRNA. In one alternative, a library or plurality of cDNAs may be screened to identify those which specifically bind a regulatory, nontranslated sequence.

Ribozymes, enzymatic RNA molecules, may also be used to catalyze the specific cleavage of RNA. The mechanism of ribozyme action involves sequence-specific hybridization of the ribozyme molecule to complementary target RNA followed by endonucleolytic cleavage at sites such as GUA, GUU, and GUC. Once such sites are identified, an oligonucleotide with the same sequence may be evaluated for secondary structural features which would render the oligonucleotide inoperable. The suitability of candidate targets may also be evaluated by testing their hybridization with complementary oligonucleotides using ribonuclease protection assays.

Complementary nucleic acids and ribozymes of the invention may be prepared via recombinant expression, in vitro or in vivo, or using solid phase phosphoramidite chemical synthesis. In addition, RNA molecules may be modified to increase intracellular stability and half-life by addition of flanking sequences at the 5′ and/or 3′ ends of the molecule or by the use of phosphorothioate or 2′ O-methyl rather than phosphodiesterase linkages within the backbone of the molecule. Modification is inherent in the production of PNAs and can be extended to other nucleic acid molecules. Either the inclusion of nontraditional bases such as inosine, queosine, and wybutosine, or the modific ati on of adenine, cyti dine, guanine, thymine, and uri dine with acetyl-, methyl-, thio-groups renders the molecule more resistant to endogenous endonucleases.

RNA Interference

RNA interference (RNAi), also known as double-stranded RNA (dsRNA)-induced gene silencing, is a method of interfering with the transcription of specific mRNAs through the production of small RNAs (siRNAs) and short hairpin RNAs (shRNAs). These RNAs are naturally formed in a multicomponent nuclease complex (RISC) in the presence of an RNAse III family nuclease (Dicer), and they serve as a guide to identify and destroy complementary transcripts. Transient infection of cells with RNAs capable of interference can bypass the need for Dicer and result in silencing of a gene for the lifespan of the introduced RNAs, usually from about 2 to about 7 days. See Paddison and Hannon (2002) Cancer Cell 2:17-23.

The RNAi pathway is believed to have evolved in early eukaryotes as a cell-based immunity against viral and genetic parasites. It is considered, however, to have great potential as a method of identifying gene function particularly in diseases such as cancer, as well as providing a highly specific means for nucleic acid-based therapies for cancer and other disorders.

cDNA Therapeutics

The cDNAs of the invention can be used in gene therapy. cDNAs can be delivered ex vivo to target cells, such as cells of bone marrow. Once stable integration and transcription and or translation are confirmed, the bone marrow may be reintroduced into the subject. Expression of the protein encoded by the cDNA may correct a disorder associated with mutation of a normal sequence, reduction or loss of an endogenous target protein, or overepression of an endogenous or mutant protein. Alternatively, cDNAs may be delivered in vivo using vectors such as retrovirus, adenovirus, adeno-associated virus, herpes simplex virus, and bacterial plasmids. Non-viral methods of gene delivery include cationic liposomes, polylysine conjugates, artificial viral envelopes, and direct injection of DNA (Anderson (1998) Nature 392:25-30; Dachs et al. (1997) Oncol Res 9:313-325; Chu et al. (1998) J Mol Med 76(3-4):184-192; Weiss et al. (1999) Cell Mol Life Sci 55(3):334-358; Agrawal (1996) Antisense Therapeutics, Humana Press, Totowa N.J.; and August et al. (1997) Gene Therapy (Advances in Pharmacology, Vol. 40), Academic Press, San Diego Calif.).

Monoclonal Antibody Therapeutics

Antibodies, and in particular monoclonal antibodies, that specifically bind a particular protein, enzyme, or receptor and block its overexpression are now being used therapeutically. The first widely accepted therapeutic antibody was HERCEPTIN (Trastuzumab, Genentech, S. San Francisco Calif.). HERCEPTIN is a humanized antibody approved for the treatment of HER2 positive metastatic breast cancer. It is designed to bind and block the function of overexpressed HER2 protein. Other monoclonal antibodies are in various stages of clinical trials for indications such as prostate cancer, lymphoma, melanoma, pneumococcal infections, rheumatoid arthritis, psoriasis, systemic lupus erythematosus, and the like.

Screening and Purification Assays

A cDNA encoding RADCP may be used to screen a library or a plurality of molecules or compounds for specific binding affinity. The libraries may be antisense molecules, artificial chromosome constructions, branched nucleic acid molecules, DNA molecules, peptides, peptide nucleic acid, proteins such as transcription factors, enhancers, or repressors, RNA molecules, ribozymes, and other ligands which regulate the activity, replication, transcription, or translation of the endogenous gene. The assay involves combining a polynucleotide with a library or plurality of molecules or compounds under conditions allowing specific binding, and detecting specific binding to identify at least one molecule which specifically binds the cDNA.

In one embodiment, the cDNA of the invention may be incubated with a plurality of purified molecules or compounds and binding activity determined by methods well known in the art, e.g., a gel-retardation assay (U.S. Pat. No. 6,010,849) or a reticulocyte lysate transcriptional assay. In another embodiment, the cDNA may be incubated with nuclear extracts from biopsied and/or cultured cells and tissues. Specific binding between the cDNA and a molecule or compound in the nuclear extract is initially determined by gel shift assay and may be later confirmed by recovering and raising antibodies against that molecule or compound. When these antibodies are added into the assay, they cause a supershift in the gel-retardation assay.

In another embodiment, the cDNA may be used to purify a molecule or compound using affinity chromatography methods well known in the art. In one embodiment, the cDNA is chemically reacted with cyanogen bromide groups on a polymeric resin or gel. Then a sample is passed over and reacts with or binds to the cDNA. The molecule or compound which is bound to the cDNA may be released from the cDNA by increasing the salt concentration of the flow-through medium and collected.

In a further embodiment, the protein or a portion thereof may be used to purify a ligand from a sample. A method for using a protein to purify a ligand would involve combining the protein with a sample under conditions to allow specific binding, detecting specific binding between the protein and ligand, recovering the bound protein, and using a chaotropic agent to separate the protein from the purified ligand.

In a preferred embodiment, RADCP may be used to screen a plurality of molecules or compounds in any of a variety of screening assays. The portion of the protein employed in such screening may be free in solution, affixed to an abiotic or biotic substrate (e.g. borne on a cell surface), or located intracellularly. For example, in one method, viable or fixed prokaryotic host cells that are stably transformed with recombinant nucleic acids that have expressed and positioned a peptide on their cell surface can be used in screening assays. The cells are screened against a plurality or libraries of ligands, and the specificity of binding or formation of complexes between the expressed protein and the ligand can be measured. Depending on the particular kind of molecules or compounds being screened, the assay may be used to identify agonists, antagonists, antibodies, DNA molecules, small drug molecules, immunoglobulins, inhibitors, mimetics, peptides, peptide nucleic acids, proteins, and RNA molecules or any other ligand, which specifically binds the protein.

In one aspect, this invention contemplates a method for high throughput screening using very small assay volumes and very small amounts of test compound as described in U.S. Pat. No. 5,876,946, incorporated herein by reference. This method is used to screen large numbers of molecules and compounds via specific binding. In another aspect, this invention also contemplates the use of competitive drug screening assays in which neutralizing antibodies capable of binding the protein specifically compete with a test compound capable of binding to the protein. Molecules or compounds identified by screening may be used in a mammalian model system to evaluate their toxicity or therapeutic potential.

Pharmaceutical Compositions

Pharmaceutical compositions may be formulated and administered, to a subject in need of such treatment, to attain a therapeutic effect. Such compositions contain the instant protein, agonists, antagonists, small drug molecules, immunoglobulins, inhibitors, mimetics, multispecific molecules, peptides, peptide nucleic acids, pharmaceutical agent, proteins, and RNA molecules. Compositions may be manufactured by conventional means such as mixing, dissolving, granulating, dragee-making, levigating, emulsifying, encapsulating, entrapping, or lyophilizing. The composition may be provided as a salt, formed with acids such as hydrochloric, sulfuric, acetic, lactic, tartaric, malic, and succinic, or as a lyophilized powder which may be combined with a sterile buffer such as saline, dextrose, or water. These compositions may include auxiliaries or excipients which facilitate processing of the active compounds.

Auxiliaries and excipients may include coatings, fillers or binders including sugars such as lactose, sucrose, mannitol, glycerol, or sorbitol; starches from corn, wheat, rice, or potato; proteins such as albumin, gelatin and collagen; cellulose in the form of hydroxypropylmethyl-cellulose, methyl cellulose, or sodium carboxymethylcellulose; gums including arabic and tragacanth; lubricants such as magnesium stearate or talc; disintegrating or solubilizing agents such as the, agar, alginic acid, sodium alginate or cross-linked polyvinyl pyrrolidone; stabilizers such as carbopol gel, polyethylene glycol, or titanium dioxide; and dyestuffs or pigments added for identify the product or to characterize the quantity of active compound or dosage.

These compositions may be administered by any number of routes including oral, intravenous, intramuscular, intra-arterial, intramedullary, intrathecal, intraventricular, transdermal, subcutaneous, intraperitoneal, intranasal, enteral, topical, sublingual, or rectal.

The route of administration and dosage will determine formulation; for example, oral administration may be accomplished using tablets, pills, dragees, capsules, liquids, gels, syrups, slurries, or suspensions; parenteral administration may be formulated in aqueous, physiologically compatible buffers such as Hanks' solution, Ringer's solution, or physiologically buffered saline. Suspensions for injection may be aqueous, containing viscous additives such as sodium carboxymethyl cellulose or dextran to increase the viscosity, or oily, containing lipophilic solvents such as sesame oil or synthetic fatty acid esters such as ethyl oleate or triglycerides, or liposomes. Penetrants well known in the art are used for topical or nasal administration.

Toxicity and Therapeutic Efficacy

A therapeutically effective dose refers to the amount of active ingredient which ameliorates symptoms or condition. For any compound, a therapeutically effective dose can be estimated from cell culture assays using normal and neoplastic cells or in animal models. Therapeutic efficacy, toxicity, concentration range, and route of administration may be determined by standard pharmaceutical procedures using experimental animals.

The therapeutic index is the dose ratio between therapeutic and toxic effects—LD50 (the dose lethal to 50% of the population)/ED50 (the dose therapeutically effective in 50% of the population)—and large therapeutic indices are preferred. Dosage is within a range of circulating concentrations, includes an ED50 with little or no toxicity, and varies depending upon the composition, method of delivery, sensitivity of the patient, and route of administration. Exact dosage will be determined by the practitioner in light of factors related to the subject in need of the treatment.

Dosage and administration are adjusted to provide active moiety that maintains therapeutic effect. Factors for adjustment include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular composition.

Normal dosage amounts may vary from 0.1 μg, up to a total dose of about 1 g, depending upon the route of administration. The dosage of a particular composition may be lower when administered to a patient in combination with other agents, drugs, or hormones. Guidance as to particular dosages and methods of delivery is provided in the pharmaceutical literature. Further details on techniques for formulation and administration may be found in the latest edition of Remington's Pharmaceutical Sciences (Mack Publishing, Easton Pa.).

Model Systems

Animal models may be used as bioassays where they exhibit a phenotypic response similar to that of humans and where exposure conditions are relevant to human exposures. Mammals are the most common models, and most infectious agent, cancer, drug, and toxicity studies are performed on rodents such as rats or mice because of low cost, availability, lifespan, gestation period, numbers of progeny, and abundant reference literature. Inbred and outbred rodent strains provide a convenient model for investigation of the physiological consequences of under- or over-expression of genes of interest and for the development of methods for diagnosis and treatment of diseases. A mammal inbred to over-express a particular gene (for example, secreted in milk) may also serve as a convenient source of the protein expressed by that gene.

Toxicology

Toxicology is the study of the effects of agents on living systems. The majority of toxicity studies are performed on rats or mice. Observation of qualitative and quantitative changes in physiology, behavior, homeostatic processes, and lethality in the rats or mice are used to generate a toxicity profile and to assess consequences on human health following exposure to the agent.

Genetic toxicology identifies and analyzes the effect of an agent on the rate of endogenous, spontaneous, and induced genetic mutations. Genotoxic agents usually have common chemical or physical properties that facilitate interaction with nucleic acids and are most harmful when chromosomal aberrations are transmitted to progeny. Toxicological studies may identify agents that increase the frequency of structural or functional abnormalities in the tissues of the progeny if administered to either parent before conception, to the mother during pregnancy, or to the developing organism. Mice and rats are most frequently used in these tests because their short reproductive cycle allows the production of the numbers of organisms needed to satisfy statistical requirements.

Acute toxicity tests are based on a single administration of an agent to the subject to determine the symptomology or lethality of the agent. Three experiments are conducted: 1) an initial dose-range-finding experiment, 2) an experiment to narrow the range of effective doses, and 3) a final experiment for establishing the dose-response curve.

Subchronic toxicity tests are based on the repeated administration of an agent. Rat and dog are commonly used in these studies to provide data from species in different families. With the exception of carcinogenesis, there is considerable evidence that daily administration of an agent at high-dose concentrations for periods of three to four months will reveal most forms of toxicity in adult animals.

Chronic toxicity tests, with a duration of a year or more, are used to test whether long term administration may elicit toxicity, teratogenesis, or carcinogenesis. When studies are conducted on rats, a minimum of three test groups plus one control group are used, and animals are examined and monitored at the outset and at intervals throughout the experiment.

Transgenic Animal Models

Transgenic rodents that over-express or under-express a gene of interest may be inbred and used to model human diseases or to test therapeutic or toxic agents. (See, e.g., U.S. Pat. No. 5,175,383 and U.S. Pat. No. 5,767,337.) In some cases, the introduced gene may be activated at a specific time in a specific tissue type during fetal or postnatal development. Expression of the transgene is monitored by analysis of phenotype, of tissue-specific mRNA expression, or of serum and tissue protein levels in transgenic animals before, during, and after challenge with experimental drug therapies.

Embryonic Stem Cells

Embryonic (ES) stem cells isolated from rodent embryos retain the ability to form embryonic tissues. When ES cells are placed inside a carrier embryo, they resume normal development and contribute to tissues of the live-born animal. ES cells are the preferred cells used in the creation of experimental knockout and knockin rodent strains. Mouse ES cells, such as the mouse 129/SvJ cell line, are derived from the early mouse embryo and are grown under culture conditions well known in the art. Vectors used to produce a transgenic strain contain a disease gene candidate and a marker gene, the latter serves to identify the presence of the introduced disease gene. The vector is transformed into ES cells by methods well known in the art, and transformed ES cells are identified and microinjected into mouse cell blastocysts such as those from the C57BL/6 mouse strain. The blastocysts are surgically transferred to pseudopregnant dams, and the resulting chimeric progeny are genotyped and bred to produce heterozygous or homozygous strains.

ES cells derived from human blastocysts may be manipulated in vitro to differentiate into at least eight separate cell lineages. These lineages are used to study the differentiation of various cell types and tissues in vitro, and they include endoderm, mesoderm, and ectodermal cell types which differentiate into, for example, neural cells, hematopoietic lineages, and cardiomyocytes.

Knockout Analysis

In gene knockout analysis, a region of a gene is enzymatically modified to include a non-mammalian gene such as the neomycin phosphotransferase gene (neo; Capecchi (1989) Science 244:1288-1292). The modified gene is transformed into cultured ES cells and integrates into the endogenous genome by homologous recombination. The inserted sequence disrupts transcription and translation of the endogenous gene. Transformed cells are injected into rodent blastulae, and the blastulae are implanted into pseudopregnant dams. Transgenic progeny are crossbred to obtain homozygous inbred lines which lack a functional copy of the mammalian gene. In one example, the mammalian gene is a human gene.

Knockin Analysis

ES cells can be used to create knockin humanized animals (pigs) or transgenic animal models (mice or rats) of human diseases. With knockin technology, a region of a human gene is injected into animal ES cells, and the human sequence integrates into the animal cell genome. Transformed cells are injected into blastulae and the blastulae are implanted as described above. Transgenic progeny or inbred lines are studied and treated with pharmaceutical agents to obtain information on treatment of the analogous human condition. These methods have been used to model several human diseases.

Non-Human Primate Model

The field of animal testing deals with data and methodology from basic sciences such as physiology, genetics, chemistry, pharmacology and statistics. These data are paramount in evaluating the effects of therapeutic agents on non-human primates as they can be related to human health. Monkeys are used as human surrogates in vaccine and drug evaluations, and their responses are relevant to human exposures under similar conditions. Cynomolgus and Rhesus monkeys (Macaca fascicularis and Macaca mulatta, respectively) and Common Marmosets (Callithrix jacchus) are the most common non-human primates (NHPs) used in these investigations. Since great cost is associated with developing and maintaining a colony of NHPs, early research and toxicological studies are usually carried out in rodent models. In studies using behavioral measures such as drug addiction, NHPs are the first choice test animal. In addition, NHPs and individual humans exhibit differential sensitivities to many drugs and toxins and can be classified as a range of phenotypes from “extensive metabolizers” to “poor metabolizers” of these agents.

In additional embodiments, the cDNAs which encode RADCP may be used in any molecular biology techniques that have yet to be developed, provided the new techniques rely on properties of cDNAs that are currently known, including, but not limited to, such properties as the triplet genetic code and specific base pair interactions.

### EXAMPLES

The examples below are provided to illustrate the subject invention and are not included for the purpose of limiting the invention. For purposes of example, preparation of the human tumorous ovary tissue (OVARTUT05) library will be described.

I cDNA Library Construction

The tissue for the OVARTUT05 library was obtained from a 62 year-old Caucasian female (specimen #0598A). Pathology indicated a grade 4 endometnoid carcinoma with extensive squamous differentiation forming a solid mass in the right ovary. The frozen tissue was homogenized and lysed in TRIZOL reagent (1 g tissue/10 ml; Invitrogen) using a POLYTRON homogenizer (Brinkmann Instruments, Westbury N.Y.). After a brief incubation on ice, chloroform was added (1:5 v/v), and the lysates were centrifuged. The upper chloroform layer was removed to a fresh tube, and the RNA was extracted with isopropanol, resuspended in DEPC-treated water, and treated with DNAse for 25 min at 37° C. The RNA was re-extracted twice with acid phenol-chloroform, pH 4.7, and precipitated using 0.3M sodium acetate and 2.5 volumes ethanol. mRNA was isolated with the OLIGOTEX kit (Qiagen, Chatsworth Calif.) and used to construct the cDNA library.

The mRNA was handled according to the recommended protocols in the SUPERSCRIPT plasmid system for cDNA synthesis and plasmid cloning (Invitrogen). The cDNAs were fractionated on a SEPHAROSE CL4B column (APB), and those cDNAs exceeding 400 bp were ligated into pINCY plasmid (Incyte Genomics). The plasmid was subsequently transformed into DH5α competent cells (Invitrogen).

II Construction of pINCY Plasmid

The plasmid was constructed by digesting the pSPORT1 plasmid (Invitrogen) with EcoRI restriction enzyme (New England Biolabs, Beverly Mass.) and filling the overhanging ends using Klenow enzyme (New England Biolabs) and 2′-deoxynucleotide 5′-triphosphates (dNTPs). The plasmid was self-ligated and transformed into the bacterial host, E. coli strain JM109.

An intermediate plasmid produced by the bacteria (pSPORT 1-ΔR1) showed no digestion with EcoRI and was digested with Hind III (New England Biolabs) and the overhanging ends were again filled in with Klenow and dNTPs. A linker sequence was phosphorylated, ligated onto the 5′ blunt end, digested with EcoRI, and self-ligated. Following transformation into JM109 host cells, plasmids were isolated and tested for preferential digestibility with EcoRI, but not with Hind III. A single colony that met this criteria was designated pINCY plasmid.

After testing the plasmid for its ability to incorporate cDNAs from a library prepared using NotI and EcoRI restriction enzymes, several clones were sequenced; and a single clone containing an insert of approximately 0.8 kb was selected from which to prepare a large quantity of the plasmid. After digestion with NotI and EcoRI, the plasmid was isolated on an agarose gel and purified using a QIAQUICK column (Qiagen) for use in library construction.

III Isolation and Sequencing of cDNA Clones

Plasmid DNA was released from the cells and purified using either the MINIPREP kit (Edge Biosystems, Gaithersburg Md.) or the REAL PREP 96 plasmid kit (Qiagen). This kit consists of a 96-well block with reagents for 960 purifications. The recommended protocol was employed except for the following changes: 1) the bacteria were inoculated into 1 ml of sterile TERRIFIC BROTH (BD Biosciences, San Jose Calif.) with carbenicillin at 25 mg/l and glycerol at 0.4%; 2) after being cultured for 19 hours, the cells were lysed with 0.3 ml of lysis buffer precipitated with isopropanol; and 3) the plasmid DNA pellet was resuspended in 0.1 ml of distilled water. After the last step in the protocol, samples were transferred to a 96-well block for storage at 4C.

The cDNAs were prepared for sequencing using the MICROLAB 2200 system (Hamilton) in combination with DNA ENGINE thermal cyclers (MJ Research). The cDNAs were sequenced by the method of Sanger and Coulson (1975; J Mol Biol 94:441-448) using a 3700, 377 or 373 DNA sequencing systems (ABI) or the MEGABACE 1000 DNA sequencing system (APB). Most of the isolates were sequenced according to standard ABI protocols and kits with solution volumes of 0.25×-1.0×concentrations. In the alternative, cDNAs were sequenced using APB solutions and dyes.

IV Extension of cDNA Sequences

The cDNAs were extended using the cDNA clone and oligonucleotide primers. One primer was synthesized to initiate 5′ extension of the known fragment, and the other, to initiate 3′ extension of the known fragment. The initial primers were designed LASERGENE software (DNASTAR) to be about 22 to 30 nucleotides in length, to have a GC content of about 50% or more, and to anneal to the target sequence at temperatures of about 68C to about 72C. Any stretch of nucleotides that would result in hairpin structures and primer-primer dimerizations was avoided.

Selected cDNA libraries were used as templates to extend the sequence. If more than one extension was necessary, additional or nested sets of primers were designed. Preferred libraries have been size-selected to include larger cDNAs and random primed to contain more sequences with 5′ or upstream regions of genes. Genomic libraries are used to obtain regulatory elements, especially extension into the 5′ promoter binding region.

High fidelity amplification was obtained by PCR using methods such as that taught in U.S. Pat. No. 5,932,451. PCR was performed in 96-well plates using the DNA ENGINE thermal cycler (MJ Research). The reaction mix contained DNA template, 200 mmol of each primer, reaction buffer containing Mg²⁺, (NH₄)₂SO₄, and β-mercaptoethanol, Taq DNA polymerase (APB), ELONGASE enzyme (Invitrogen), and Pfu DNA polymerase (Stratagene), with the following parameters for primer pair PCI A and PCI B (Incyte Genomics): Step 1: 94C, three min; Step 2: 94C, 15 sec; Step 3: 60C, one min; Step 4: 68C, two min; Step 5: Steps 2, 3, and 4 repeated 20 times; Step 6: 68C, five min; Step 7: storage at 4C. In the alternative, the parameters for primer pair T7 and SK+ (Stratagene) were as follows: Step 1: 94C, three min; Step 2: 94C, 15 sec; Step 3: 57C, one min; Step 4: 68C, two min; Step 5: Steps 2, 3, and 4 repeated 20 times; Step 6: 68C, five min; Step 7: storage at 4C.

The concentration of DNA in each well was determined by dispensing 100 μl PICOGREEN quantitation reagent (0.25% reagent in 1×TE, v/v; Molecular Probes) and 0.5 μl of undiluted PCR product into each well of an opaque fluorimeter plate (Corning Life Sciences, Acton Mass.) and allowing the DNA to bind to the reagent. The plate was scanned in a Fluoroskan II (Labsystems Oy) to measure the fluorescence of the sample and to quantify the concentration of DNA. A 5 μl to 10 μl aliquot of the reaction mixture was analyzed by electrophoresis on a 1% agarose mini-gel to determine which reactions were successful in extending the sequence.

The extended clones were desalted, concentrated, transferred to 384-well plates, digested with CviJI cholera virus endonuclease (Molecular Biology Research, Madison Wis.), and sonicated or sheared prior to religation into pUC18 vector (APB). For shotgun sequences, the digested nucleotide sequences were separated on low concentration (0.6 to 0.8%) agarose gels, fragments were excised, and the agar was digested with AGARACE enzyme (Promega). Extended clones were religated using T4 DNA ligase (New England Biolabs) into pUC18 vector (APB), treated with Pfu DNA polymerase (Stratagene) to fill-in restriction site overhangs, and transfected into E. coli competent cells. Transformed cells were selected on antibiotic-containing media, and individual colonies were picked and cultured overnight at 37C in 384-well plates in LB/2×carbenicillin liquid media.

The cells were lysed, and DNA was amplified using primers, Taq DNA polymerase (APB) and Pfu DNA polymerase (Stratagene) with the following parameters: Step 1: 94C, three min; Step 2: 94C, 15 sec; Step 3: 60C, one min; Step 4: 72C, two min; Step 5: steps 2, 3, and 4 repeated 29 times; Step 6: 72C, five min; Step 7: storage at 4C. DNA was quantified using PICOGREEN quantitative reagent (Molecular Probes) as described above. Samples with low DNA recoveries were reamplified using the conditions described above. Samples were diluted with 20% dimethylsulfoxide (DMSO; 1:2, v/v), and sequenced using DYENAMIC energy transfer sequencing primers and the DYENAMIC DIRECT cycle sequencing kit (APB) or the ABI PRISM BIGDYE terminator cycle sequencing kit (PE Biosystems).

V Homology Searching of cDNA Clones and Their Deduced Proteins

The cDNAs of the Sequence Listing or their deduced amino acid sequences were used to query databases such as GenBank, SwissProt, BLOCKS, and the like. These databases that contain previously identified and annotated sequences or domains were searched using BLAST or BLAST 2 (Altschul et al. supra; Altschul, supra) to produce alignments and to determine which sequences were exact matches or homologs. The alignments were to sequences of prokaryotic (bacterial) or eukaryotic (animal, fungal, or plant) origin. Alternatively, algorithms such as the one described in Smith and Smith (1992, Protein Engineering 5:35-51) could have been used to deal with primary sequence patterns and secondary structure gap penalties. All of the sequences disclosed in this application have lengths of at least 49 nucleotides, and no more than 12% uncalled bases (where N is recorded rather than A, C, G, or T).

As detailed in Karlin (supra), BLAST matches between a query sequence and a database sequence were evaluated statistically and only reported when they satisfied the threshold of 10⁻²⁵ for nucleotides and 10⁻¹⁴ for peptides. Homology was also evaluated by product score calculated as follows: the % nucleotide or amino acid identity [between the query and reference sequences] in BLAST is multiplied by the % maximum possible BLAST score [based on the lengths of query and reference sequences] and then divided by 100. In comparison with hybridization procedures used in the laboratory, the electronic stringency for an exact match was set at 70, and the conservative lower limit for an exact match was set at approximately 40 (with 1-2% error due to uncalled bases).

The BLAST software suite, freely available sequence comparison algorithms (NCBI, Bethesda Md.), includes various sequence analysis programs including “blastn” that is used to align nucleic acid molecules and BLAST 2 that is used for direct pairwise comparison of either nucleic or amino acid molecules. BLAST programs are commonly used with gap and other parameters set to default settings, e.g.: Matrix: BLOSUM62; Reward for match: 1; Penalty for mismatch: 2; Open Gap: 5 and Extension Gap: 2 penalties; Gap x drop-off: 50; Expect: 10; Word Size: 11; and Filter: on. Identity is measured over the entire length of a sequence or some smaller portion thereof. Brenner et al. (1998; Proc Natl Acad Sci 95:6073-6078, incorporated herein by reference) analyzed the BLAST for its ability to identify structural homologs by sequence identity and found 30% identity is a reliable threshold for sequence alignments of at least 150 residues and 40%, for alignments of at least 70 residues.

The mammalian cDNAs of this application were compared with assembled consensus sequences or templates found in the LIFESEQ GOLD database. Component sequences from cDNA, extension, full length, and shotgun sequencing projects were subjected to PHRED analysis and assigned a quality score. All sequences with an acceptable quality score were subjected to various pre-processing and editing pathways to remove low quality 3′ ends, vector and linker sequences, polyA tails, Alu repeats, mitochondrial and ribosomal sequences, and bacterial sequences. Edited sequences had to be at least 50 bp in length, and low-information sequences and repetitive elements such as dinucleotide repeats, Alu repeats, and the like, were replaced by “Ns” or masked.

Edited sequences were subjected to assembly procedures in which the sequences were assigned to gene bins. Each sequence could only belong to one bin, and sequences in each bin were assembled to produce a template. Newly sequenced components were added to existing bins using BLAST and CROSSMATCH. To be added to a bin, the component sequences had to have a BLAST quality score greater than or equal to 150 and an alignment of at least 82% local identity. The sequences in each bin were assembled using PHRAP. Bins with several overlapping component sequences were assembled using DEEP PHRAP. The orientation of each template was determined based on the number and orientation of its component sequences.

Bins were compared to one another and those having local similarity of at least 82% were combined and reassembled. Bins having templates with less than 95% local identity were split. Templates were subjected to analysis by STITCHER/EXON MAPPER algorithms that analyze the probabilities of the presence of splice variants, alternatively spliced exons, splice junctions, differential expression of alternative spliced genes across tissue types or disease states, and the like. Assembly procedures were repeated periodically, and templates were annotated using BLAST against GenBank databases such as GBpri. An exact match was defined as having from 95% local identity over 200 base pairs through 100% local identity over 100 base pairs and a homolog match as having an E-value (or probability score) of ≦1×10⁻⁸. The templates were also subjected to frameshift FASTx against GENPEPT, and homolog match was defined as having an E-value of ≦1×10⁻⁸. Template analysis and assembly was described in U.S. Ser. No. 09/276,534, filed Mar. 25, 1999.

Following assembly, templates were subjected to BLAST, motif, and other functional analyses and categorized in protein hierarchies using methods described in U.S. Ser. No. 08/812,290 and U.S. Ser. No. 08/811,758, both filed Mar. 6, 1997; in U.S. Ser. No. 08/947,845, filed Oct. 9, 1997; and in U.S. Ser. No. 09/034,807, filed Mar. 4, 1998. Then templates were analyzed by translating each template in all three forward reading frames and searching each translation against the PFAM database of hidden Markov model-based protein families and domains using the HMMER software package (Washington University School of Medicine, St. Louis Mo.).

The cDNA was further analyzed using MACDNASIS PRO software (Hitachi Software Engineering), and LASERGENE software (DNASTAR) and queried against public databases such as the GenBank rodent, mammalian, vertebrate, prokaryote, and eukaryote databases, SwissProt, BLOCKS, PRINTS, PFAM, and Prosite.

VI Chromosome Mapping

Radiation hybrid and genetic mapping data available from public resources such as the Stanford Human Genome Center (SHGC), Whitehead Institute for Genome Research (WIGR), and Genethon are used to determine if any of the cDNAs presented in the Sequence Listing have been mapped. Any of the fragments of the cDNAs encoding RADCP that have been mapped result in the assignment of all related regulatory and coding sequences mapping to the same location. The genetic map locations are described as ranges, or intervals, of human chromosomes. The map position of an interval, in cM (which is roughly equivalent to 1 megabase of human DNA), is measured relative to the terminus of the chromosomal p-arm.

VII Hybridization Technologies and Analyses

Donor Tissues

The matched normal/tumor lung tissues used in the lung microarray experiments were obtained from the Roy Castle International Centre for Lung Cancer Research (RCIC), Liverpool UK. Descriptions of the donor tissue is found in the table below. The first column of the table shows donor ID; the second column, the location of the tumor; the third column, diagnosis; and the fourth column, the percentage of the tumor composed of tumor cells.

| Donor | | | |
|---|---|---|---|
| ID | Location of Lung Tumor | Diagnosis | % tumor cells |
| 5792 | Left Lung, Upper Lobe | Squamous Cell CA | NA |
| 7173 | Right Lung | Squamous Cell CA | 70 |
| 7176 | Right Lung Middle and | Adenosquamous CA | 60 |
| | Lower Lobe | | |
| 7178 | Left Lung, Upper Lobe | Squamous CA | 70 |
| 7191 | Left Lung | Squamous Cell CA | 70 |
| 7964 | Lung Tumor | Non-Small Cell | 70 |
| | | Adenocarcinoma | |

Immobilization of cDNAs on a Substrate

The cDNAs are applied to a substrate by one of the following methods. A mixture of cDNAs is fractionated by gel electrophoresis and transferred to a nylon membrane by capillary transfer. Alternatively, the cDNAs are individually ligated to a vector and inserted into bacterial host cells to form a library. The cDNAs are then arranged on a substrate by one of the following methods. In the first method, bacterial cells containing individual clones are robotically picked and arranged on a nylon membrane. The membrane is placed on LB agar containing selective agent (carbenicillin, kanamycin, ampicillin, or chloramphenicol depending on the vector used) and incubated at 37C for 16 hr. The membrane is removed from the agar and consecutively placed colony side up in 10% SDS, denaturing solution (1.5 M NaCl, 0.5 M NaOH), neutralizing solution (1.5 M NaCl, 1 M Tris, pH 8.0), and twice in 2×SSC for 10 min each. The membrane is then UV irradiated in a STRATALINKER UV-crosslinker (Stratagene).

In the second method, cDNAs are amplified from bacterial vectors by thirty cycles of PCR using primers complementary to vector sequences flanking the insert. PCR amplification increases a starting concentration of 1-2 ng nucleic acid to a final quantity greater than 5 μg. Amplified nucleic acids from about 400 bp to about 5000 bp in length are purified using SEPHACRYL-400 beads (APB). Purified nucleic acids are arranged on a nylon membrane manually or using a dot/slot blotting manifold and suction device and are immobilized by denaturation, neutralization, and UV irradiation as described above. Purified nucleic acids are robotically arranged and immobilized on polymer-coated glass slides using the procedure described in U.S. Pat. No. 5,807,522. Polymer-coated slides are prepared by cleaning glass microscope slides (Corning Life Sciences) by ultrasound in 0.1% SDS and acetone, etching in 4% hydrofluoric acid (VWR Scientific Products, West Chester Pa.), coating with 0.05% aminopropyl silane (Sigma-Aldrich) in 95% ethanol, and curing in a 110C oven. The slides are washed extensively with distilled water between and after treatments. The nucleic acids are arranged on the slide and then immobilized by exposing the array to UV irradiation using a STRATALINKER UV-crosslinker (Stratagene). Arrays are then washed at room temperature in 0.2% SDS and rinsed three times in distilled water. Non-specific binding sites are blocked by incubation of arrays in 0.2% casein in phosphate buffered saline (PBS; Tropix, Bedford Mass.) for 30 min at 60C; then the arrays are washed in 0.2% SDS and rinsed in distilled water as before.

Probe Preparation for Membrane Hybridization

Hybridization probes derived from the cDNAs of the Sequence Listing are employed for screening cDNAs, mRNAs, or genomic DNA in membrane-based hybridizations. Probes are prepared by diluting the cDNAs to a concentration of 40-50 ng in 45 μl TE buffer, denaturing by heating to 10° C. for five min, and briefly centrifuging. The denatured cDNA is then added to a REDIPRIME tube (APB), gently mixed until blue color is evenly distributed, and briefly centrifuged. Five μl of [³²P]dCTP is added to the tube, and the contents are incubated at 37C for 10 min. The labeling reaction is stopped by adding 5 μl of 0.2M EDTA, and probe is purified from unincorporated nucleotides using a PROBEQUANT G-50 microcolumn (APB). The purified probe is heated to 100C for five min, snap cooled for two min on ice, and used in membrane-based hybridizations as described below.

Probe Preparation for Polymer Coated Slide Hybridization

Hybridization probes derived from mRNA isolated from samples are employed for screening cDNAs of the Sequence Listing in array-based hybridizations. Probe is prepared using the GEMbright kit (Incyte Genomics) by diluting mRNA to a concentration of 200 ng in 9 μl TE buffer and adding 5 μl 5×buffer, 1 μl 0.1 M DTT, 3 μl Cy3 or Cy5 labeling mix, 1 μl RNAse inhibitor, 1 μl reverse transcriptase, and 5 μl 1×yeast control mRNAs. Yeast control mRNAs are synthesized by in vitro transcription from noncoding yeast genomic DNA (W Lei, unpublished). As quantitative controls, one set of control mRNAs at 0.002 ng, 0.02 ng, 0.2 ng, and 2 ng are diluted into reverse transcription reaction mixture at ratios of 1:100,000, 1:10,000, 1:1000, and 1:100 (w/w) to sample mRNA respectively. To examine mRNA differential expression patterns, a second set of control mRNAs are diluted into reverse transcription reaction mixture at ratios of 1:3, 3:1, 1:10, 10:1, 1:25, and 25:1 (w/w). The reaction mixture is mixed and incubated at 37C for two hr. The reaction mixture is then incubated for 20 min at 85C, and probes are purified using two successive CHROMA SPIN+TE 30 columns (Clontech, Palo Alto Calif.). Purified probe is ethanol precipitated by diluting probe to 90 μl in DEPC-treated water, adding 2 μl 1 mg/ml glycogen, 60 μl 5 M sodium acetate, and 300 μl 100% ethanol. The probe is centrifuged for 20 min at 20,800×g, and the pellet is resuspended in 12 μl resuspension buffer, heated to 65C for five min, and mixed thoroughly. The probe is heated and mixed as before and then stored on ice. Probe is used in high density array-based hybridizations as described below.

Membrane-Based Hybridization

Membranes are pre-hybridized in hybridization solution containing 1% Sarkosyl and 1×high phosphate buffer (0.5 M NaCl, 0.1 M Na₂HPO₄, 5 mM EDTA, pH 7) at 55C for two hr. The probe, diluted in 15 ml fresh hybridization solution, is then added to the membrane. The membrane is hybridized with the probe at 55C for 16 hr. Following hybridization, the membrane is washed for 15 min at 25C in 1 mM Tris (pH 8.0), 1% Sarkosyl, and four times for 15 min each at 25C in 1 mM Tris (pH 8.0). To detect hybridization complexes, XOMAT-AR film (Eastman Kodak, Rochester N.Y.) is exposed to the membrane overnight at −70C, developed, and examined.

Polymer Coated Slide-Based Hybridization

Probe is heated to 65C for five min, centrifuged five min at 9400 rpm in a 5415C microcentrifuge (Eppendorf Scientific, Westbury N.Y.), and then 18 μl is aliquoted onto the array surface and covered with a coverslip. The arrays are transferred to a waterproof chamber having a cavity just slightly larger than a microscope slide. The chamber is kept at 100% humidity internally by the addition of 140 μl of 5×SSC in a corner of the chamber. The chamber containing the arrays is incubated for about 6.5 hr at 60C. The arrays are washed for 10 min at 45C in 1×SSC, 0.1% SDS, and three times for 10 min each at 45C in 0.1×SSC, and dried.

Hybridization reactions are performed in absolute or differential hybridization formats. In the absolute hybridization format, probe from one sample is hybridized to array elements, and signals are detected after hybridization complexes form. Signal strength correlates with probe mRNA levels in the sample. In the differential hybridization format, differential expression of a set of genes in two biological samples is analyzed. Probes from the two samples are prepared and labeled with different labeling moieties. A mixture of the two labeled probes is hybridized to the array elements, and signals are examined under conditions in which the emissions from the two different labels are individually detectable. Elements on the array that are hybridized to substantially equal numbers of probes derived from both biological samples give a distinct combined fluorescence (Shalon WO95/35505).

Hybridization complexes are detected with a microscope equipped with an Innova 70 mixed gas 10 W laser (Coherent, Santa Clara Calif.) capable of generating spectral lines at 488 nm for excitation of Cy3 and at 632 nm for excitation of Cy5. The excitation laser light is focused on the array using a 20×microscope objective (Nikon, Melville N.Y.). The slide containing the array is placed on a computer-controlled X-Y stage on the microscope and raster-scanned past the objective with a resolution of 20 micrometers. In the differential hybridization format, the two fluorophores are sequentially excited by the laser. Emitted light is split, based on wavelength, into two photomultiplier tube detectors (PMT R1477, Hamamatsu Photonics Systems, Bridgewater N.J.) corresponding to the two fluorophores. Appropriate filters positioned between the array and the photomultiplier tubes are used to filter the signals. The emission maxima of the fluorophores used are 565 nm for Cy3 and 650 nm for Cy5. The sensitivity of the scans is calibrated using the signal intensity generated by the yeast control mRNAs added to the probe mix. A specific location on the array contains a complementary DNA sequence, allowing the intensity of the signal at that location to be correlated with a weight ratio of hybridizing species of 1:100,000.

The output of the photomultiplier tube is digitized using a 12-bit RTI-835H analog-to-digital (AID) conversion board (Analog Devices, Norwood Mass.) installed in an IBM-compatible PC computer. The digitized data are displayed as an image where the signal intensity is mapped using a linear 20-color transformation to a pseudocolor scale ranging from blue (low signal) to red (high signal). The data is also analyzed quantitatively. Where two different fluorophores are excited and measured simultaneously, the data are first corrected for optical crosstalk (due to overlapping emission spectra) between the fluorophores using the emission spectrum for each fluorophore. A grid is superimposed over the fluorescence signal image such that the signal from each spot is centered in each element of the grid. The fluorescence signal within each element is then integrated to obtain a numerical value corresponding to the average intensity of the signal. The software used for signal analysis was the GEMTOOLS program (Incyte Genomics).

Results

The Human Genome GEM series 1 (HG1) microarray (Incyte Genomics) was used in the studies summarized in the table below. HG1 contains 9,766 array elements which represent 7,612 annotated clusters and 1,382 unannotated clusters. Log₂ values >1.5 indicate highly significant differential expression of RADCP in these experiments. The first column of the table shows the log₂ (Cy3/Cy5) ratio; the second column, the description of the Cy3 sample; the third column, the description of the Cy3 sample; and the fourth column, the donor ID. As can be seen on lines 2 and 3, 4 and 5 of the table, the experiments using donors 4625 and 7191, respectively were repeated with highly concordant results.

| log₂(Cy3/ | | | |
|---|---|---|---|
| Cy5) | Cy3 Sample | Cy5 Sample* | Donor |
| 2.28464 | PBMC Cells Pool, | PBMC Cells Pool, t/Dex | 4625 |
| | Untx 24 hr | 1 microM 3 d | |
| 2.031216 | PBMC Cells Pool, | PBMC Cells Pool, t/LPS + | 4625 |
| | Untx 24 hr | Dex 1 mcg/mL, 1 μM 3 d | |
| 2.002371 | Normal Lung, Left | Lung Tumor, Left, SC CA | 7191 |
| | Lobe | | |
| 1.938059 | Normal Lung, Left | Lung Tumor, Left Lobe, SC | 7191 |
| | Lobe | CA | |
| 1.988685 | Normal Lung, Right | Lung Tumor, Right, SC CA | 7173 |
| | Lobe | | |
| 1.666069 | Normal Lung | Lung Tumor, SC CA | 5792 |
| 1.631694 | Normal Lung, Right | Lung Tumor, Right Lobe, | 7176 |
| | Lobe | Adenosquamous CA | |
| 1.54463 | Normal Lung, Left | Lung Tumor, Left Upper | 7178 |
| | Upper Lobe | Lobe, SC CA | |
| 1.534728 | Normal Lung | Lung Tumor, NSC Lung | 7964 |
| | | AdenoCA | |

*Abbreviations: Untx = untreated, Dex = Dexamethasone, LPS = lipopolysaccharide, CA = carcinoma, SC = squamous cell, and NSC = non-small cell

As shown above, RADCP is differentially expressed in PMBC treated with dexamethazone +LPS and in lung tumors tissue with matched normals from the same donor.

VIII Northern Analysis, Transcript Imaging, and Guilt-By-Association

Northern Analysis

Northern analysis is a laboratory technique used to detect the presence of a transcript of a gene and involves the hybridization of a labeled nucleotide sequence to a membrane on which RNAs from a particular cell type or tissue have been bound. The technique is described in EXAMPLE VII above and in Ausubel, supra, units 4.1-4.9)

Analogous computer techniques applying BLAST are used to search for identical or related molecules in nucleotide databases such as GenBank or the LIFESEQ database (Incyte Genomics). This analysis is faster than multiple membrane-based hybridizations. In addition, the sensitivity of the computer search can be modified to determine whether any particular match is categorized as exact or homologous. The basis of the search is the product score which was described above.

The description and results of transcript imaging, one form of electronic northern analysis, is described and presented below.

Transcript Imaging

A transcript image was performed for RADCP using the LIFESEQ GOLD database (Incyte Genomics). This process assessed the relative abundance of the expressed polynucleotides in all of the cDNA libraries and was described in U.S. Pat. No. 5,840,484, incorporated herein by reference. All sequences and cDNA libraries in the LIFESEQ database are categorized by system, organ/tissue and cell type. The categories include cardiovascular system, connective tissue, digestive system, embryonic structures, endocrine system, exocrine glands, female and male genitalia, germ cells, hemic/immune system, liver, musculoskeletal system, nervous system, pancreas, respiratory system, sense organs, skin, stomatognathic system, unclassified/mixed, and the urinary tract. Criteria for transcript imaging are selected from category, number of cDNAs per library, library description, disease indication, clinical relevance of sample, and the like.

For each category, the number of libraries in which the sequence was expressed are counted and shown over the total number of libraries in that category. For each library, the number of cDNAs are counted and shown over the total number of cDNAs in that library. In some transcript images, all enriched, normalized (NORM) or subtracted (SUB) libraries, which have high copy number sequences can be removed prior to processing, and all mixed or pooled tissues, which are considered non-specific in that they contain more than one tissue type or more than one subject's tissue, can be excluded from the analysis. Treated and untreated cell lines and/or fetal tissue data can also be excluded where clinical relevance is emphasized. Conversely, fetal tissue can be emphasized wherever elucidation of inherited disorders or differentiation of particular adult or embryonic stem cells into tissues or organs (such as heart, kidney, nerves or pancreas) would be aided by removing clinical samples from the analysis.

Tables 1A and 1B show the northern analysis for RADCP produced using the LIFESEQ Gold database (Incyte Genomics, Palo Alto Calif.). In Table 1A, the first column presents the tissue categories; the second column, the number of cDNAs in the tissue category; the third column, the number of libraries in which at least one transcript was found; the fourth column, absolute abundance of the transcript; and the fifth column, percent abundance of the transcript. As can be seen in Table 1A, RADCP and its encoding transcript were most highly expressed in the hemic and immune category.

| Category | cDNAs | Found | Abund | % Abund |
|---|---|---|---|---|
| Cardiovascular | 253105 | 1/64 | 1 | 0.0004 |
| Connective Tissue | 134008 | 2/41 | 2 | 0.0015 |
| Digestive | 447016 | 12/130 | 13 | 0.0029 |
| Embryonic Tissue | 106591 | 1/21 | 1 | 0.0009 |
| Endocrine System | 210781 | 7/50 | 8 | 0.0038 |
| Exocrine Glands | 252458 | 8/61 | 8 | 0.0032 |
| Reproductive, Female | 392343 | 7/92 | 9 | 0.0023 |
| Reproductive, Male | 430286 | 6/109 | 8 | 0.0019 |
| Germ Cells | 36677 | 2/5 | 5 | 0.0136 |
| Hemic and Immune | 662225 | 55/153 | 128 | 0.0193 |
| Liver | 92176 | 1/25 | 7 | 0.0076 |
| Musculoskeletal | 154504 | 4/44 | 4 | 0.0026 |
| Nervous System | 904527 | 15/185 | 18 | 0.0020 |
| Pancreas | 100545 | 4/21 | 4 | 0.0040 |
| Respiratory | 362922 | 9/83 | 13 | 0.0036 |
| Sense Organs | 19253 | 0/8 | 0 | 0.0000 |
| Skin | 72082 | 1/15 | 1 | 0.0014 |
| Stomatognathic | 10988 | 0/4 | 0 | 0.0000 |
| Unclassified/Mixed | 103494 | 2/8 | 7 | 0.0068 |
| Urinary Tract | 252077 | 2/57 | 2 | 0.0008 |
| Totals | 4998058 | 139/1176 | 239 | 0.0048 |

Table 1B shows expression of RADCP in samples from subjects with a cell proliferative or immunological disorder. The first column lists the library name, the second column, the number of cDNAs sequenced for that library; the third column, the description of the tissue; the fourth column, the absolute abundance of the transcript; and the fifth column, the percent abundance of the transcript.

| Library | cDNAs | Description | Abund | % Abund |
|---|---|---|---|---|
| SPLNNOP01 | 305 | spleen, 23 M, TIGR | 8 | 2.6230 |
| EOSINOT02 | 2357 | probable periph blood, | 5 | 0.2121 |
| | | eosinophils, asthma, M/F | | |
| EOSITXT01 | 8987 | periph blood, eosino- | 10 | 0.1113 |
| | | phils, t/IL-5 | | |
| EOSINOT03 | 3730 | periph blood, eosino- | 2 | 0.0536 |
| | | phils, asthma, M/F | | |
| EOSIHET02 | 9312 | periph blood, eosino- | 4 | 0.0430 |
| | | phils, hypereosinophilia, | | |
| | | 48 M | | |
| MPHGNOT03 | 7817 | periph blood, macro- | 11 | 0.1407 |
| | | phages, adher PBMC, | | |
| | | M/F | | |
| NEUTGMT01 | 6482 | periph blood, granulo- | 7 | 0.1080 |
| | | cytes, M/F, t/GM-CSF | | |
| NEUTLPT01 | 5609 | periph blood, granulo- | 5 | 0.0891 |
| | | cytes, M/F, t/LPS | | |
| NEUTFMT01 | 5577 | periph blood, granulo- | 3 | 0.0538 |
| | | cytes, M/F, t/fMLP | | |
| SPLNNOT09 | 4095 | spleen, Gaucher's, 22 M | 2 | 0.0488 |
| THYMDIT01 | 7027 | thymus, hyperplasia, aw/ | 2 | 0.0285 |
| | | myasthenia gravis, 16 F | | |

As can be seen from the table above, RADCP was significantly expressed in hemic and immune cells and particularly in asthma, thymus hyperplasia and hypereosinophilia. It must also be noted that RADCP was not expressed in the EOSINOT01 library which had 2404 cDNAs and was made from pooled peripheral blood, specifically eosinophils of nonallergic male and female subjects.

Guilt-By-Association

GBA identifies cDNAs that are expressed in a plurality of cDNA libraries relating to a specific disease process, subcellular compartment, cell type, tissue type, or species. The expression patterns of cDNAs with unknown function are compared with the expression patterns of genes having well documented function to determine whether a specified co-expression probability threshold is met. Through this comparison, a subset of the cDNAs having a highly significant co-expression probability with the known genes are identified.

The cDNAs originate from human cDNA libraries from any cell or cell line, tissue, or organ and may be selected from a variety of sequence types including, but not limited to, expressed sequence tags (ESTs), assembled polynucleotides, full length gene coding regions, promoters, introns, enhancers, 5′ untranslated regions, and 3′ untranslated regions. To have statistically significant analytical results, the cDNAs need to be expressed in at least five cDNA libraries. The number of cDNA libraries whose sequences are analyzed can range from as few as 500 to greater than 10,000.

The method for identifying cDNAs that exhibit a statistically significant co-expression pattern is as follows. First, the presence or absence of a gene in a cDNA library is defined: a gene is present in a library when at least one fragment of its sequence is detected in a sample taken from the library, and a gene is absent from a library when no corresponding fragment is detected in the sample.

Second, the significance of co-expression is evaluated using a probability method to measure a due-to-chance probability of the co-expression. The probability method can be the Fisher exact test, the chi-squared test, or the kappa test. These tests and examples of their applications are well known in the art and can be found in standard statistics texts (Agresti (1990) Categorical Data Analysis, John Wiley & Sons, New York N.Y.; Rice (1988) Mathematical Statistics and Data Analysis, Duxbury Press, Pacific Grove Calif.). A Bonferroni correction (Rice, supra, p. 384) can also be applied in combination with one of the probability methods for correcting statistical results of one gene versus multiple other genes. In a preferred embodiment, the due-to-chance probability is measured by a Fisher exact test, and the threshold of the due-to-chance probability is set preferably to less than 0.001.

This method of estimating the probability for co-expression of two genes assumes that the libraries are independent and are identically sampled. However, in practical situations, the selected cDNA libraries are not entirely independent because: 1) more than one library may be obtained from a single subject or tissue, and 2) different numbers of cDNAs, typically ranging from 5,000 to 10,000, may be sequenced from each library. In addition, since a Fisher exact co-expression probability is calculated for each gene versus every other gene that occurs in at least five libraries, a Bonferroni correction for multiple statistical tests is used (See Walker et al. (1999; Genome Res 9:1198-203; expressly incorporated herein by reference).

IX Complementary Molecules

Molecules complementary to the cDNA, from about 5 (PNA) to about 5000 bp (complement of a cDNA insert), are used to detect or inhibit gene expression. These molecules are selected using LASERGENE software (DNASTAR). Detection is described in Example VII. To inhibit transcription by preventing promoter binding, the complementary molecule is designed to bind to the most unique 5′ sequence and includes nucleotides of the 5′ UTR upstream of the initiation codon of the open reading frame. Complementary molecules include genomic sequences (such as enhancers or introns) and are used in “triple helix” base pairing to compromise the ability of the double helix to open sufficiently for the binding of polymerases, transcription factors, or regulatory molecules. To inhibit translation, a complementary molecule is designed to prevent ribosomal binding to the mRNA encoding the mammalian protein.

Complementary molecules are placed in expression vectors and used to transform a cell line to test efficacy; into an organ, tumor, synovial cavity, or the vascular system for transient or short term therapy; or into a stem cell, zygote, or other reproducing lineage for long term or stable gene therapy. Transient expression lasts for a month or more with a non-replicating vector and for three months or more if appropriate elements for inducing vector replication are used in the transformation/expression system.

Stable transformation of appropriate dividing cells with a vector encoding the complementary molecule produces a transgenic cell line, tissue, or organism (U.S. Pat. No. 4,736,866). Those cells that assimilate and replicate sufficient quantities of the vector to allow stable integration also produce enough complementary molecules to compromise or entirely eliminate activity of the cDNA encoding the mammalian protein.

X Expression of RADCP

Expression and purification of the mammalian protein are achieved using either a mammalian cell expression system or an insect cell expression system. The pUB6/V5-His vector system (Invitrogen) is used to express RADCP in CHO cells. The vector contains the selectable bsd gene, multiple cloning sites, the promoter/enhancer sequence from the human ubiquitin C gene, a C-terminal V5 epitope for antibody detection with anti-V5 antibodies, and a C-terminal polyhistidine (6×His) sequence for rapid purification on PROBOND resin (Invitrogen). Transformed cells are selected on media containing blasticidin.

Spodoptera frugiperda (Sf9) insect cells are infected with recombinant Autographica californica nuclear polyhedrosis virus (baculovirus). The polyhedrin gene is replaced with the mammalian cDNA by homologous recombination and the polyhedrin promoter drives cDNA transcription. The protein is synthesized as a fusion protein with 6×his which enables purification as described above. Purified protein is used in the following activity and to make antibodies.

XI Production of Antibodies

RADCP are purified using polyacrylamide gel electrophoresis and used to immunize mice or rabbits. Antibodies are produced using the protocols below. Alternatively, the amino acid sequences of RADCP are analyzed using LASERGENE software (DNASTAR) to determine regions of high antigenicity. An antigenic epitope, usually found near the C-terminus or in a hydrophilic region is selected, synthesized, and used to raise antibodies. Typically, epitopes of about 15 residues in length are produced using an ABI 431A peptide synthesizer (Applied Biosystems) using Fmoc-chemistry and coupled to KLH (Sigma-Aldrich) by reaction with N-maleimidobenzoyl-N-hydroxysuccinimide ester to increase antigenicity.

Rabbits are immunized with the epitope-KLH complex in complete Freund's adjuvant. Immunizations are repeated at intervals thereafter in incomplete Freund's adjuvant. After a minimum of seven weeks for mouse or twelve weeks for rabbit, antisera are drawn and tested for antipeptide activity. Testing involves binding the peptide to plastic, blocking with 1% bovine serum albumin, reacting with rabbit antisera, washing, and reacting with radio-iodinated goat anti-rabbit IgG. Methods well known in the art are used to determine antibody titer and the amount of complex formation.

XII Immunopurification of Naturally Occurring Protein Using Antibodies

Naturally occurring or recombinant protein is purified by immunoaffinity chromatography using antibodies which specifically bind the protein. An immunoaffinity column is constructed by covalently coupling the antibody to CNBr-activated SEPHAROSE resin (APB). Media containing the protein is passed over the immunoaffinity column, and the column is washed using high ionic strength buffers in the presence of detergent to allow preferential absorbance of the protein. After coupling, the protein is eluted from the column using a buffer of pH 2-3 or a high concentration of urea or thiocyanate ion to disrupt antibody/protein binding, and the protein is collected.

XIII Western Analysis

Electrophoresis and Blotting

Samples containing protein are mixed in 2×loading buffer, heated to 95 C for 3-5 min, and loaded on 4-12% NUPAGE Bis-Tris precast gel (Invitrogen). Unless indicated, equal amounts of total protein are loaded into each well. The gel is electrophoresced in 1×MES or MOPS running buffer (Invitrogen) at 200 V for approximately 45 min on an Xcell II apparatus (Invitrogen) until the RAINBOW marker (APB) has resolved, and dye front approaches the bottom of the gel. The gel and its supports are removed from the apparatus and soaked in 1×transfer buffer (Invitrogen) with 10% methanol for a few minutes; and the PVDF membrane is soaked in 100% methanol for a few seconds to activate it. The membrane, gel, and supports are placed on the TRANSBLOT SD transfer apparatus (Biorad, Hercules Calif.) and a constant current of 350 mAmps is applied for 90 min.

Conjugation with Antibody and Visualization

After the proteins are transferred to the membrane, it is blocked in 5% (w/v) non-fat dry milk in 1×phosphate buffered saline (PBS) with 0.1% Tween 20 detergent (blocking buffer) on a rotary shaker for at least 1 hr at room temperature or at 4C overnight. After blocking, the buffer is removed, and 10 ml of primary antibody in blocking buffer is added. The membrane is incubated on the rotary shaker for 1 hr at room temperature or overnight at 4C. The membrane is washed 3×for 10 min each with PBS-Tween (PBST), and secondary antibody, conjugated to horseradish peroxidase, is added at a 1:3000 dilution in 10 ml blocking buffer. The membrane and solution are shaken for 30 min at room temperature and then washed three times for 10 min each with PBST.

The wash solution is carefully removed, and the membrane is moistened with ECL+ chemilum-inescent detection system (APB) and incubated for approximately 5 min. The membrane, protein side down, is placed on BIOMAX M film (Eastman Kodak) and developed for approximately 30 seconds.

XIV Antibody Arrays

Protein:Protein Interactions

In an alternative to yeast two hybrid system analysis of proteins, an antibody array can be used to study protein-protein interactions and phosphorylation. A variety of protein ligands are immobilized on a membrane using methods well known in the art. The array is incubated in the presence of cell lysate until protein:antibody complexes are formed. Proteins of interest are identified by exposing the membrane to an antibody specific to the protein of interest. In the alternative, a protein of interest is labeled with digoxigenin (DIG) and exposed to the membrane; then the membrane is exposed to anti-DIG antibody which reveals where the protein of interest forms a complex. The identity of the proteins with which the protein of interest interacts is determined by the position of the protein of interest on the membrane.

Proteomic Profiles

Antibody arrays can also be used for high-throughput screening of recombinant antibodies. Bacteria containing antibody genes are robotically-picked and gridded at high density (up to 18,342 different double-spotted clones) on a filter. Up to 15 antigens at a time are used to screen for clones to identify those that express binding antibody fragments. These antibody arrays can also be used to identify proteins which are differentially expressed in samples (de Wildt, supra)

XV Screening Molecules for Specific Binding with the cDNA or Protein

The cDNA, or fragments thereof, or the protein, or portions thereof, are labeled with ³²P-dCTP, Cy3-dCTP, or Cy5-dCTP (APB), or with BIODIPY or FITC (Molecular Probes, Eugene Oreg.), respectively. Libraries of candidate molecules or compounds previously arranged on a substrate are incubated in the presence of labeled cDNA or protein. After incubation under conditions for either a nucleic acid or amino acid sequence, the substrate is washed, and any position on the substrate retaining label, which indicates specific binding or complex formation, is assayed, and the ligand is identified. Data obtained using different concentrations of the nucleic acid or protein are used to calculate affinity between the labeled nucleic acid or protein and the bound molecule.

XVI Two-Hybrid Screen

A yeast two-hybrid system, MATCHMAKER LexA Two-Hybrid system (Clontech Laboratories, Palo Alto Calif.), is used to screen for peptides that bind the mammalian protein of the invention. A cDNA encoding the protein is inserted into the multiple cloning site of a pLexA vector, ligated, and transformed into E. coli. cDNA, prepared from mRNA, is inserted into the multiple cloning site of a pB42AD vector, ligated, and transformed into E. coli to construct a cDNA library. The pLexA plasmid and pB42AD-cDNA library constructs are isolated from E. coli and used in a 2:1 ratio to co-transform competent yeast EGY48[p8op-lacZ] cells using a polyethylene glycol/lithium acetate protocol. Transformed yeast cells are plated on synthetic dropout (SD) media lacking histidine (-His), tryptophan (-Trp), and uracil (-Ura), and incubated at 30C until the colonies have grown up and are counted. The colonies are pooled in a minimal volume of 1×TE (pH 7.5), replated on SD/-His/-Leu/-Trp/-Ura media supplemented with 2% galactose (Gal), 1% raffinose (Raf), and 80 mg/ml 5-bromo-4-chloro-3-indolyl β-d-galactopyranoside (X-Gal), and subsequently examined for growth of blue colonies. Interaction between expressed protein and cDNA fusion proteins activates expression of a LEU2 reporter gene in EGY48 and produces colony growth on media lacking leucine (-Leu). Interaction also activates expression of β-galactosidase from the p8op-lacZ reporter construct that produces blue color in colonies grown on X-Gal.

Positive interactions between expressed protein and cDNA fusion proteins are verified by isolating individual positive colonies and growing them in SD/-Trp/-Ura liquid medium for 1 to 2 days at 30C. A sample of the culture is plated on SD/-Trp/-Ura media and incubated at 30C until colonies appear. The sample is replica-plated on SD/-Trp/-Ura and SD/-His/-Trp/-Ura plates. Colonies that grow on SD containing histidine but not on media lacking histidine have lost the pLexA plasmid. Histidine-requiring colonies are grown on SD/Gal/Raf/X-Gal/-Trp/-Ura, and white colonies are isolated and propagated. The pB42AD-cDNA plasmid, which contains a cDNA encoding a protein that physically interacts with the mammalian protein, is isolated from the yeast cells and characterized.

XVII RADCP Assay

RADCP is assayed for Ras binding by the method of Vavvas (supra). RADCP is expressed as a GST fusion protein and the GST-RADCP fusion is incubated with Ras in the presence of either GTPyS or GDPβS. Glutathione-SEPHAROSE beads (APB) are added to recover the GST-RADCP fusion and GST-RADCP-Ras complexes from solution. Proteins are eluted from the glutathione-SEPHAROSE beads with SDS sample buffer and separated by SDS-PAGE. Following electrophoresis, proteins are transferred to a PVDF membrane (APB) and probed for Ras with monoclonal anti-Ras antibodies.

All patents and publications mentioned in the specification are incorporated by reference herein. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the field of molecular biology or related fields are intended to be within the scope of the following claims.

What is claimed is:

1. A purified protein comprising a polypeptide having the amino acid sequence of SEQ ID NO:1.

2. A biologically active portion of the protein of claim 1 wherein the portion extends from residue A119 to residue T211 of SEQ ID NO:1.

3. An antigenic determinant of the protein of claim 1 wherein the determinant extends from residue F30 to residue M75 of SEQ ID NO:1.

4. A composition comprising the protein of claim 1 and a labeling moiety.

5. A composition comprising the protein of claim 1 and a pharmaceutical carrier.

6. A substrate upon which the protein of claim 1 is immobilized.

7. An array element comprising the protein of claim 1.

8. A method for detecting expression of a protein having the amino acid sequence of SEQ ID NO:1 in a sample, the method comprising:
a) performing an assay to determine the amount of the protein of claim 1 in a sample; and
b) comparing the amount of protein to standards, thereby detecting expression of the protein in the sample.

9. The method of claim 8 wherein the assay is selected from antibody or protein arrays, enzyme-linked immunosorbent assays, fluorescence-activated cell sorting, spatial immobilization such as 2D-PAGE and scintillation counting, high performance liquid chromatography, or mass spectrophotometry, radioimmunoassays and western analysis.

10. The method of claim 8 wherein the sample is from blood, lung, lymph node, prostate, spleen, tonsil, and thymus.

11. The method of claim 8 wherein the protein is differentially expressed when compared with at least one standard and is diagnostic of a cell proliferative disorder.

12. A method for using a protein to screen a plurality of molecules and compounds to identify at least one ligand, the method comprising:
a) combining the protein of claim 1 with a plurality of molecules and compounds under conditions to allow specific binding; and
b) detecting specific binding, thereby identifying a ligand that specifically binds the protein.

13. The method of claim 12 wherein the molecules and compounds are selected from agonists, antagonists, antibodies, DNA molecules, small drug molecules, multispecific molecules, peptides, pharmaceutical agents, proteins, and RNA molecules.

14. A method for using a protein to identify an antibody that specifically binds the protein having the amino acid sequence of SEQ ID NO:1 comprising:
a) contacting a plurality of antibodies with the protein of claim 1 under conditions to allow specific binding, and
b) detecting specific binding between an antibody and the protein, thereby identifying an antibody that specifically binds the protein having the amino acid sequence of SEQ ID NO:1.

15. The method of claim 14, wherein the plurality of antibodies are selected from a polyclonal antibody, a monoclonal antibody, a chimeric antibody, a recombinant antibody, a humanized antibody, a single chain antibody, a Fab fragment, an F(ab′)₂ fragment, an Fv fragment; and an antibody-peptide fusion protein.

16. A method of using a protein to prepare and purify a polyclonal antibody comprising:
a) immunizing a animal with a protein of claim 1 under conditions to elicit an antibody response;
b) isolating animal antibodies;
c) attaching the protein to a substrate;
d) contacting the substrate with isolated antibodies under conditions to allow specific binding to the protein; and
e) dissociating the antibodies from the protein, thereby obtaining purified polyclonal antibodies.

17. A method of using a protein to prepare a monoclonal antibody comprising:
a) immunizing a animal with a protein of claim 1 under conditions to elicit an antibody response;
b) isolating antibody-producing cells from the animal;
c) fusing the antibody-producing cells with immortalized cells in culture to form monoclonal antibody producing hybridoma cells;
d) culturing the hybridoma cells; and
e) isolating from culture monoclonal antibody that specifically binds the protein.

18. A method for using a protein to diagnose a cancer comprising:
a) performing an assay to quantify the expression of the protein of claim 1 in a sample; and
b) comparing the expression of the protein to standards, thereby diagnosing a cell proliferative disorder.

19. The method of claim 18 wherein the sample is selected from blood, lung, lymph node, prostate, spleen, tonsil, and thymus.

20. A method for testing a molecule or compound for effectiveness as an agonist comprising:
a) exposing a sample comprising the protein of claim 1 to the molecule or compound; and
b) detecting agonist activity in the sample.

21. A method for testing a molecule or compound for effectiveness as an antagonist, the method comprising:
a) exposing a sample comprising the protein of claim 1 to a molecule or compound; and
b) detecting antagonist activity in the sample.

22. An isolated antibody that specifically binds a protein having the amino acid sequence of SEQ ID NO:1.

23. A polyclonal antibody produced by the method of claim 16.

24. A monoclonal antibody produced by the method of claim 17.

25. A method for using an antibody to detect expression of a protein in a sample, the method comprising:
a) combining the antibody of claim 22 with a sample under conditions which allow the formation of antibody:protein complexes; and
b) detecting complex formation, wherein complex formation indicates expression of the protein in the sample.

26. The method of claim 25 wherein the sample is from blood, lung, lymph node, prostate, spleen, tonsil, and thymus.

27. The method of claim 25 wherein complex formation is compared with standards and is diagnostic of a cell proliferative disorder.

28. A method for using an antibody to immunopurify a protein comprising:
a) attaching the antibody of claim 22 to a substrate;
b) exposing the antibody to a sample containing protein under conditions to allow antibody:protein complexes to form;
c) dissociating the protein from the complex; and
d) collecting the purified protein.

29. A composition comprising an antibody of claim 22 and a labeling moiety.

30. A kit comprising the composition of claim 29.

31. An array element comprising the antibody of claim 22.

32. A substrate upon which the antibody of claim 22 is immobilized.

33. A composition comprising an antibody of claim 22 and a pharmaceutical agent.

34. The composition of claim 33 wherein the composition is lyophilized.

35. A method for using a composition to assess efficacy of a molecule or compound, the method comprising:
a) treating a sample containing protein with a molecule or compound;
b) contacting the protein in the sample with the composition of claim 33 under conditions for complex formation;
c) determining the amount of complex formation; and
d) comparing the amount of complex formation in the treated sample with the amount of complex formation in an untreated sample, wherein a difference in complex formation indicates efficacy of the molecule or compound.

36. A method for using a composition to assess toxicity of a molecule or compound, the method comprising:
a) treating a sample containing protein with a molecule or compound;
b) contacting the protein in the sample with the composition of claim 33 under conditions for complex formation;
c) determining the amount of complex formation; and
d) comparing the amount of complex formation in the treated sample with the amount of complex formation in an untreated sample, wherein a difference in complex formation indicates toxicity of the molecule or compound.

37. A method for treating a cancer comprising administering to a subject in need of therapeutic intervention the antibody of claim 22.

38. A method for treating a cancer comprising administering to a subject in need of therapeutic intervention the antibody of claim 22.

39. A method for treating a cancer comprising administering to a subject in need of therapeutic intervention the composition of claim 33.

40. A method for delivering a therapeutic agent to a cell comprising:
a) attaching the therapeutic agent to a multispecific molecule identified by the method of claim 12; and
b) administering the multispecific molecule to a subject in need of therapeutic intervention, wherein the multispecific molecule specifically binds the protein having the amino acid sequence of SEQ ID NO:1 thereby delivering the therapeutic agent to the cell.

41. The method of claim 40, wherein the cell is an epithelial cell of the lung.

42. An agonist that specifically binds the protein of claim 1.

43. A composition comprising an agonist of claim 42 and a pharmaceutical carrier.

44. An antagonist that specifically binds the protein of claim 1.

45. A composition comprising the antagonist of claim 44 and a pharmaceutical carrier.

46. A pharmaceutical agent that specifically binds the protein of claim 1.

47. A composition comprising the pharmaceutical agent of claim 46 and a pharmaceutical carrier.

48. A small drug molecule that specifically binds the protein of claim 1.

49. A composition comprising the small drug molecule of claim 48 and a pharmaceutical carrier.

49. An antisense molecule of 18 to 30 nucleotides in length that specifically binds a portion of a polynucleotide having a nucleic acid sequence of SEQ ID NO:1 wherein the antisense molecule inhibits expression of the protein encoded by the polynucleotide.

50. The antisense molecule of claim 49 wherein the antisense molecule comprises at least one modified internucleoside linkage.

51. The antisense molecule of claim 50 wherein the modified internucleoside linkage is a phosphorothioate linkage.

52. The antisense molecule of claim 49 wherein the antisense molecule comprises at least one nucleotide analog.

53. The antisense molecule of claim 52 wherein the modified nucleobase is a 5-methylcytosine.
